(19) United States
(12) Patent Application Publication
(10) Pub. No.: US 2010/0107473 A1
(43) Pub. Date: May 6, 2010

(54) ENHANCEMENT OF COLD TOLERANCE IN PLANTS

(75) Inventors: Chunsheng Zhang, Summerville, SC (US); Kimberly Ann Winkeler, Summerville, SC (US); Samantha Abigail Miller, summerville, SC (US); Teresa Vales, Summerville, SC (US); Kirk Foutz, Summerville, SC (US); Yuan Zhao, Summerville, SC (US); Marion Wood, Summerville, SC (US)

Correspondence Address: FOLEY AND LARDNER LLP;SUITE 500, 3000 K STREET NW, WASHINGTON, DC, 20007, US

(73) Assignee: ARBORGEN LLC

(21) Appl. No.: 12/593,225

(22) PCT Filed: Mar. 28, 2008
(86) PCT No.: PCT/US08/04054

§ 371 Date: Jan. 20, 2010

Related U.S. Application Data
(60) Provisional application No. 60/908,940, filed on Mar. 29, 2007.

(51) Int. Cl. C10L 1/00; C12N 15/63; C12N 5/10; A01H 5/00; A01H 1/00; C09F 1/00; D21C 3/22; B27L 1/00

(52) U.S. Cl. 44/307; 435/320.1; 435/419; 800/298; 800/290; 800/289; 530/206; 162/70; 144/340

(57) ABSTRACT

Novel dehydrin promoters isolated from Eucalyptus dunnii and Eucalyptus macarthurii are cold-inducible and can be used for driving CBF genes in plants, including trees, to enhance tolerance to freezing temperatures or water stress and reduce undesirable effects associated with CBF gene expression.

### PRIORITY

The application claims priority to U.S. Provisional application 60/908,940, filed Mar. 29, 2007.

### FIELD OF THE INVENTION

The present invention relates to plant biotechnology and alteration of gene expression in transformed plants. More specifically, this invention relates to methods of enhancing stress tolerance in plants of industrial interest by regulation of expression of genes encoding C-repeat-binding factors (CBFs), while reducing undesirable effects associated with the expression of the desired genes.

### BACKGROUND OF THE INVENTION

Low temperature stress is a major environmental factor that not only limits the areas where plants can be grown, but also affects the quantity and quality of the crops. Each year, worldwide losses in crop production due to low temperature damage amount to approximately $2 billion. Occasional freezes in Florida have forced the citrus belt to be moved further south, and citrus crops in California have often sustained severe damage in recent years due to winter freeze.

Exposure of plants to water stress diminishes plant turgor, leads to leaf wilting and decreases photosynthesis, thus hampering and eventually preventing normal growth. Plant gene expression is strongly affected by wilting (Guerrero and Mullet, 1988). Desiccation during frost or water stress causes an increase in the hydrolysis of starch and proteins with a consequent reduction in enzyme activity. The degree of water loss injury depends on the conditions of the soil and atmospheric factors.

Plants vary greatly in their ability to withstand low temperature or water stress. Plants that originate in tropical regions, such as corn, rice and cassava, can be killed or severely damaged during a drought or when exposed to a low temperature, even if the temperature is above freezing. Plants that originate in temperate climates, on the other hand, are less susceptible to water stress or freezing temperatures.

Eucalyptus plants comprise more than eight hundred species which grow in the tropical and temperate regions of the world. Eucalyptus has a high growth rate, adapts to wide range of environments, and displays little susceptibility to insect damage. In addition to its exceptional growth properties, Eucalyptus trees provide the largest source of fibers for the paper industry. Fibers from hardwood species, such as Eucalyptus, are generally much shorter than fibers from softwoods, such as pine. The shorter fibers produced from Eucalyptus result in the production of pulp and paper with desirable surface characteristics, including smoothness and brightness, but low tear or tensile strength. Eucalyptus timber is used for plywood and particleboard and the lumber has economical importance in the furniture and flooring industries and provides a source of firewood and ornamental and construction materials, such as beams and poles. Wood chips from Eucalyptus can be used in a variety of composite lumber products, such as oriented strand board (OSB) or medium density fiberboard (MDF). As a fast growing species, Eucalyptus is also used as a fuelwood, for charcoal, and for additional energy production applications such as a feedstock for biofuels and bioproducts manufacture. Eucalyptus is also grown to produce mulch and provide windbreaks for aesthetic and industrial applications. Essential oils from Eucalyptus are also used for cleaning, cosmetic products, such as soaps and perfumes, as well as for medicinal purposes. Furthermore, Eucalyptus plantations are considered valuable for production of carbon-neutral and renewable biofuel, which can be used as a substitute for costly fossil fuels. Eucalyptus biomass can be converted into building materials, paper, fuels, food, animal feed and other products, such as plant-derived chemicals, like waxes and cleaners. Solid biomass may be also used to generate process heat and electric power. Biomass processing may additionally be used for biorefinery to produce fuels, chemicals, new bio-based materials, and electric power. Biofuel can be produced from Eucalyptus trees using the fast pyrolysis process, a thermochemical bioconversion method in which renewable biomass is rapidly heated to 450°-600° C. in the absence of air.

Eucalyptus is the most commonly planted hardwood in the world. However, Eucalyptus species are mostly confined to temperate areas because of their high sensitivity to low temperatures and their limited ability to withstand water stress. While some species of Eucalyptus are more tolerant than others to exposure to low temperature, sudden severe frosts pose a great threat to survival in most, if not all, Eucalyptus species. Induced tolerance to progressively lower temperatures, known as cold acclimation, may be obtained only after exposure to a hardening cold treatment accompanied by a decrease in light intensity and day length, but its success depends on several factors, including the species and its origin, duration of the hardening period and the health of the tree. The ability of most Eucalyptus species to resist water stress is also very limited. Excess water loss leads to a general decrease in growth and significant reductions in leaf area ratio, specific leaf area and leaf-to-root area ratio.

Plants that are more resistant to stress, including freezing stress and water stress, could be grown in a larger range of geographical areas and their crops would be subject to fewer environmental risks. However, despite continued efforts, traditional breeding has had only limited success in imparting crop plants with better stress tolerance.

Plant genetic engineering has great potential for the improvement of commercially important plant species. In recent years, genetic engineering of trees has been used to improve wood quality for application in the paper and fuel industry. Species in the Populus genera have served as models for the genetic engineering of trees (Kim et al. 1997), and various traits, such as insect resistance and herbicide tolerance, have been engineered into tree species (Klopfenstein et al. 1993, De Block 1990).

Several research groups in recent years have focused their research on the identification of stress-regulated genes and their functions in the mechanisms responsible for stress tolerance in plants. The genes that are induced upon cold treatment in plants are collectively named Cold-Regulated Genes (COR). In Arabidopsis, cold acclimation is associated with the induction of COR genes mediated by the cold- and dehydration-responsive DNA regulatory element designated the CRT (C-repeat)/DRE (dehydration responsive element) DNA regulatory element. A small family of cold-responsive transcriptional activators known as CBF1, CBF2 and CBF3 or DREB1 b, DREB1 c and DREB1a, respectively, recognizes the CRT (C-repeat)/DRE sequence present in the promoter regions of cold and dehydration responsive genes. Increased expression of Arabidopsis CBF1, a transcriptional activator that binds to the CRT/DRE sequence, induces COR gene expression and increases the freezing tolerance of non-acclimated Arabidopsis plants. The CBF genes are induced within 15 min after exposure of the plants to a low, nonfreezing temperature, and within two hours induction of cold-regulated genes that contain the CRT/DRE-regulatory element; known as the “CBF regulon”, takes place, leading to an increase in plant freezing tolerance over the next few days (Jaglo-Ottosen et al., 1998). The CBF-regulon expression also increases tolerance to drought and high salinity stress. (Stockinger et al., 1997; Fowler and Thomashow, 2002; Kasuga et al., 1999; Haake et al., 2002).

CBF genes have been found in several crop species, including corn, soybean, wheat, rice, barley, tomato, alfalfa, canola, as well as in vegetables, such as Brassica napus, and trees. The presence of CBF genes has been demonstrated in four different species of Eucalyptus: Eucalyptus grandis (ArborGen, egCBF1 and egCBF3); Eucalyptus dunnii (ArborGen, ed7.1 and ed8.1); Eucalyptus gunnii (GenBank Accession No. ABB51638); and Eucalyptus globulus (GenBank, Accession No. ABF70207).

In the past few years the development of transgenic plants with improved stress tolerance has received great attention. However, the expression of Arabidopsis CBF1 in transgenic plants, while improving tolerance to cold, drought and salt loading, has been accompanied by a negative effect on the growth and yield of the plants under normal growth conditions. Expression of CBF/DREB1 has been reported to produce dwarfism in Arabidopsis and tomato (Gilmour et al., 2000; Hsieh et al., 2002b; Kasuga et al., 1999; Liu et al., 1998). The introduction of a wheat DREB2A homologue gene into rice plants also results in dwarfism (Shen et al., 2003).

Thus, there is an immediate need to develop better technologies to improve stress tolerance, including freezing and water loss tolerance, in commercially important trees and plants, including tropical trees, such as Eucalyptus, Citrus, Avocado, Papaya, Nutmeg, Pistachio, Kiwi and Jojoba, that allow modification of the expression of the genes involved in stress response and cold and water loss tolerance by over-expressing transcriptional activators, such as CBF1, while reducing undesirable effects associated with the complex mechanisms that regulate stress tolerance in plants.

### SUMMARY OF THE INVENTION

It is an object of the present invention to provide genes that may be manipulated to increase stress tolerance in plants.

It is also an object of the present invention to provide promoter sequences that drive the expression of genes that increase stress tolerance in plants.

It is another object of this invention to provide transgenic plants exhibiting improved stress tolerance compared to non-transformed plants of the same species.

It is further an object of the present invention to provide methods for modifying the phenotype of plants, by altering their stress tolerance.

It is still another object of the present invention to provide methods of making wood from transgenic plants exhibiting improved stress tolerance.

It is still another object of the present invention to provide methods of making structure lumber from transgenic plants exhibiting improved stress tolerance.

It is still another object of the present invention to provide methods of making biofuel from transgenic plants exhibiting improved stress tolerance.

To accomplish these and other objectives, the present invention provides DNA constructs comprising a CBF gene sequence or a CBF homologous gene sequence represented by SEQ ID NOs: 1, 3, 5 or 7, operably linked to a stress-related promoter sequence, which, upon induction by desiccation, cold or high-salt conditions, causes expression of the CBF gene or CBF homologous gene in plants, while reducing undesirable effects associated with the expression of the CBF gene.

In one embodiment, the invention provides a tree cell transformed with a DNA construct that comprises the Arabidopsis thaliana CBF2 gene operably linked to the stress-related gene Arabidopsis thaliana promoter RD29A.

In another embodiment, the present invention provides transgenic plants that exhibit increased expression of the CBF2, as compared to the expression of CBF2 in non-transformed plants of the same species, and have a phenotype that is characterized by increased stress tolerance when compared to the phenotype of non-transformed plants of the same species. Transgenic plants may be dicotyledonous or monocotyledon plants. Preferably, the transgenic plants are angiosperm plants. More preferably, the transgenic plants are hardwood tropical trees, including eucalyptus, poplar, citrus, papaya, avocado, nutmeg, pistachio, kiwi and jojoba. Organs of transgenic plants, comprising leaves, stems, flowers, ovaries, fruits, seeds and calluses, are also included in the embodiment of the invention.

In an additional embodiment, the present invention provides methods for producing a transgenic tree comprising transforming a tree cell with DNA constructs that comprise the Arabidopsis thaliana CBF2 gene operably linked to the Arabidopsis thaliana RD29A promoter, to produce a transformed tree cell; and culturing the transformed tree cell under conditions that promote growth of a tree that exhibits improved stress tolerance compared to a non-transformed tree of the same species.

In a further embodiment, the invention provides methods for enhancing freezing tolerance in a tree comprising transforming a tree cell with a DNA construct that comprises the Arabidopsis thaliana CBF2 gene operably linked to the Arabidopsis thaliana RD29A promoter, to produce a transformed tree cell; and culturing the transformed tree cell under conditions that promote growth of a tree, wherein the polypeptide encoded by the AtCBF2 gene is expressed in the transformed tree cell, and the tree is a transgenic tree that exhibits improved cold tolerance compared to a non-transformed tree of the same species.

In still another embodiment, the invention provides methods for making wood and wood pulp from transgenic trees comprising transforming tree cells with DNA constructs that comprise the Arabidopsis thaliana CBF2 gene operably linked to the Arabidopsis thaliana RD29A promoter, to produce transformed tree cells; culturing the transformed tree cells under conditions that promote growth of a tree, wherein the polypeptide encoded by the AtCBF2 gene is expressed in the transformed tree cells, and the tree is a transgenic tree that exhibits improved stress tolerance compared to a non-transformed tree of the same species.

In yet another embodiment, the present invention provides dehydrin promoter sequences represented by SEQ ID Nos: 9 or 10, or fragments or variants thereof. comprising a nucleic acid sequence of at least 30 contiguous nucleotides, that is at least 50% identical to a dehydrin promoter having SEQ ID NOs: 9 or 10.

In another embodiment the present invention provides DNA constructs comprising a desired gene operably linked to a dehydrin promoter sequence represented by SEQ ID Nos: 9 or 10, or fragments or variants thereof comprising a nucleic acid sequence of at least 30 contiguous nucleotides, that is at least 50% identical to a dehydrin promoter having SEQ ID NOs: 9 or 10, wherein the promoter drives the expression of the desired gene in a plant upon exposure of the plant to a stress condition for a period of time, while reducing the undesirable effects associated with the expression of the desired gene. Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point.

In another embodiment the present invention provides DNA constructs comprising an isolated polynucleotide comprising a nucleic acid sequence of a CBF homologous gene represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, operably linked to a promoter, wherein the promoter drives the expression of the CBF homologous gene in a plant upon exposure of the plant to a stress condition for a period of time, while reducing the undesirable effects associated with the expression of CBF genes. Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. The nucleic acid sequences of the invention may include one or more base deletions, substitutions, insertions and/or additions.

In a further embodiment, the invention provides a plant cell transformed with a DNA construct that comprises an isolated polynucleotide comprising a nucleic acid sequence of a CBF homologous gene represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, operably linked to a promoter, wherein the promoter drives the expression of the CBF homologous gene in a plant upon exposure of the plant to a stress condition for a period of time, while reducing the undesirable effects associated with the expression of CBF genes. Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter having SEQ ID NOs: 9, 10 or 11.

In another embodiment, the present invention provides transgenic plants that exhibit increased stress tolerance, as compared to the level of stress tolerance in non-transformed plants of the same species. Preferably, the stress condition is a freezing temperature from 0° C. to −30° C. In an additional preferred aspect of the invention, the stress condition is lack of water. Transgenic plants may be dicotyledonous or monocotyledon plants. Preferably, the transgenic plants are angiosperm plants. More preferably, the transgenic plants are hardwood tropical trees, including eucalyptus, poplar, citrus, papaya, avocado, nutmeg, pistachio, kiwi and jojoba. Organs of transgenic plants, comprising leaves, stems, flowers, ovaries, fruits, seeds and calluses, are also included in the embodiment of the invention.

In an additional embodiment, the present invention provides methods for producing a transgenic plant comprising transforming a plant cell with DNA constructs that comprise an isolated polynucleotide comprising a nucleic acid sequence of a CBF homologous gene represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, operably linked to a promoter, wherein the promoter drives the expression of the CBF homologous gene in a plant upon exposure of the plant to a stress condition for a period of time, while reducing the undesirable effects associated with the expression of CBF genes, to produce a transformed plant cell; and culturing the transformed plant cell under conditions that promote growth of a plant that exhibits improved cold tolerance compared to a non-transformed plant of the same species. Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter having SEQ ID NOs: 9, 10 or 11.

In a further embodiment, the invention provides methods for enhancing freezing tolerance in an angiosperm plant comprising transforming a plant cell with a DNA construct that comprises an isolated polynucleotide comprising a nucleic acid sequence of a CBF homologous gene represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, operably linked to a promoter, wherein the promoter drives the expression of the plant transcription factor in a plant upon exposure of the plant to a stress condition for a period of time ranging from 2 hours to 72 hours, while reducing the undesirable effects associated with the expression of CBF genes, to produce a transformed plant cell; and culturing the transformed plant cell under conditions that promote growth of a plant, wherein the polypeptide encoded by the isolated polynucleotide sequence is expressed in the transformed plant cell, and the plant is a transgenic plant that exhibits improved cold tolerance compared to a non-transformed plant of the same species. Preferably, the promoter is the Arabidopsis thaliana rd29A promoter or the CaMV 35S promoter. More preferably, the promoter is a dehydrin promoter having a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. Preferably, the stress condition is a freezing temperature from 0° C. to −30° C.

In still another embodiment, the invention provides a method for making wood and/or wood pulp from a transgenic plant comprising transforming a plant cell with a DNA construct that comprises an isolated polynucleotide comprising a nucleic acid sequence of a CBF homologous gene represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, operably linked to a promoter, wherein the promoter drives the expression of the CBF homologous gene in a plant upon exposure of the plant to a stress condition for a period of time, while reducing the undesirable effects associated with the expression of CBF genes, to produce a transformed plant cell; culturing the transformed plant cell under conditions that promote growth of a plant, wherein the polypeptide encoded by the polynucleotide sequence is expressed in the transformed plant cell; and manufacturing wood from the transgenic plant. Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. Preferably, the plant is a transgenic plant that exhibits improved stress tolerance compared to a non-transformed plant of the same species.

In still another embodiment, the invention provides a method for making veneer and/or tall oil from transgenic plants comprising transforming a plant cell with a DNA construct that comprises an isolated polynucleotide comprising a nucleic acid sequence of a CBF homologous gene represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, operably linked to a promoter, wherein the promoter drives the expression of the CBF homologous gene in a plant upon exposure of the plant to a stress condition for a period of time, while reducing the undesirable effects associated with the expression of CBF genes, to produce transformed a plant cell; culturing the transformed plant cell under conditions that promote growth of a plant, wherein the polypeptide encoded by the polynucleotide sequence is expressed in the transformed plant cells, and manufacturing veneer and/or tall oil from the transgenic plant. Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. Preferably, the plant is a transgenic plant that exhibits improved stress tolerance compared to a non-transformed plant of the same species.

In an additional embodiment, the invention provides a method for producing biofuel from a transgenic plant comprising transforming a plant cell with a DNA construct that comprises an isolated polynucleotide comprising a CBF2 gene sequence or a CBF homologous gene sequence represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, operably linked to a promoter, wherein the promoter drives the expression of the plant transcription factor in a plant upon exposure of the plant to a stress condition for a period of time, while reducing the undesirable effects associated with the expression of CBF genes, to produce a transformed plant cell; culturing the transformed plant cell under conditions that promote growth of a plant, wherein the polypeptide encoded by the polynucleotide sequence is expressed in the transformed plant cell; and producing biofuel from the transgenic plant. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter having SEQ ID NOs: 9, 10 or 11. Preferably, the transgenic plant is cold-acclimated prior to exposure to the stress condition, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the phenotype of the transgenic plant is characterized by increased stress tolerance compared to a non-transformed plant of the same species.

In a further embodiment, the invention provides DNA constructs that comprise a nucleic acid sequence that encodes a polypeptide having CBF activity comprising an amino acid sequence represented by SEQ ID NOs: 2, 4, 6 or 8, or a polypeptide having CBF activity comprising an amino acid sequence which has at least 70%, more preferably at least 80%, more preferably at least 90% and most preferably at least 95% sequence identity to an amino acid sequence represented by SED ID Nos: 2, 4, 6 or 8, wherein the nucleic acid sequence is operably linked to one or more suitable promoters that cause expression of the nucleic acid sequence. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter having SEQ ID NOs: 9, 10 or 11. Polypeptides of the invention may include amino acid substitutions, additions and deletions that do not alter transcription factor activity.

In another embodiment, the invention provides an isolated plant cell expressing a polypeptide encoded by a polynucleotide comprising a CBF homologous gene sequence represented by SEQ ID NOs: 1, 3, 5 or 7, or a fragment or variant thereof comprising a nucleotide sequence which is at least 70% identical to the nucleotide sequence represented by SEQ ID NOs: 1, 3, 5 or 7, wherein the expression of the CBF homologous gene is driven by a promoter upon exposure of the plant to a stress condition for a period of time, while reducing the undesirable effects associated with the expression of CBF genes Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. Preferably, the polypeptide comprises an'amino acid sequence represented by SEQ ID NOs: 2, 4, 6 or 8, or is a polypeptide having CBF activity comprising an amino acid sequence which has at least 70%, more preferably at least 80%, more preferably at least 90% and most preferably at least 95% sequence identity to an amino acid sequence represented by SEQ ID Nos: 2, 4, 6 or 8.

In yet another embodiment, the present invention provides a transgenic plant that expresses a polypeptide that comprises an amino acid sequence represented by SEQ ID NOs: 2, 4, 6 or 8, or a polypeptide having CBF activity comprising an amino acid sequence which has at least 70%, more preferably at least 80%, more preferably at least 90% and most preferably at least 95% sequence identity to an amino acid sequence represented by SEQ ID Nos: 2, 4, 6 or 8, wherein the transgenic plant exhibits a phenotype that is different from the phenotype of a non-transformed plant of the same species, and wherein the expression of the polypeptide is driven upon exposure of the plant to a stress condition for a period of time, without undesirable effects associated with the expression of polypeptides having CBF activity Preferably, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the transgenic plant exhibits improved stress tolerance compared to a non-transformed plant of the same species.

In still another embodiment, the invention provides a method for making wood and/or wood pulp from a transgenic plant comprising transforming a plant cell with a DNA construct comprising a desired gene operably linked to one or more suitable promoters that cause expression of the desired gene, to produce a transformed plant cell; culturing the transformed plant cell under conditions that promote growth of a plant, wherein the polypeptide encoded by the desired gene is expressed in the transformed plant cell, and the plant is a transgenic plant that exhibits a phenotype that is different from the phenotype of a non-transformed plant of the same species; and manufacturing wood from the transgenic plant. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter having SEQ ID NOs: 9, 10 or 11. Preferably, the transgenic plant is cold-acclimated prior to exposure to the stress condition, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the transgenic plant exhibits improved stress tolerance compared to a non-transformed plant of the same species.

In still another embodiment, the invention provides a method for making veneer and/or tall oil from a transgenic plant comprising transforming a plant cell with a DNA construct that comprises a desired gene operably linked to one or more suitable promoters that cause expression of the desired gene, to produce a transformed plant cell; culturing the transformed plant cell under conditions that promote growth of a plant, wherein the polypeptide encoded by the desired gene is expressed in the transformed plant cell, and the plant is a transgenic plant that exhibits a phenotype that is different from the phenotype of a non-transformed plant of the same species; and manufacturing veneer and/or tall oil from the transgenic plant. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. Preferably, the transgenic plant is cold-acclimated prior to exposure to the stress condition, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the transgenic plant exhibits improved stress tolerance compared to a non-transformed plant of the same species.

In an additional embodiment, the invention provides a method for producing biofuel from a transgenic plant comprising transforming a plant cell with a DNA construct that comprises a desired gene operably linked to one or more suitable promoters that cause expression of the desired gene, to produce a transformed plant cell; culturing the transformed plant cell under conditions that promote growth of a plant, wherein the product of the desired gene is expressed in the transformed plant cells, and the plant is a transgenic plant that exhibits a phenotype that is different from the phenotype of a non-transformed plant of the same species; and producing biofuel from the transgenic plant. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. Preferably, the transgenic plant is cold-acclimated prior to exposure to the stress condition, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the phenotype of the transgenic plant is characterized by increased stress tolerance compared to a non-transformed plant of the same species.

In yet another embodiment, the invention provides a methods for producing bioenergy from a transgenic plant comprising transforming a plant cell with a DNA construct that comprises a desired gene operably linked to one or more suitable promoters that cause expression of the desired gene, to produce a transformed plant cell; culturing the transformed plant cell under conditions that promote growth of a plant, wherein the product of the desired gene is expressed in the transformed plant cell, and the plant is a transgenic plant that exhibits a phenotype that is different from the phenotype of a non-transformed plant of the same species; and producing bioenergy from the transgenic plants. Preferably, the promoter is the Arabidopsis thaliana rd29A promoter or the CaMV 35S promoter. More preferably, the promoter is a dehydrin promoter comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter comprising SEQ ID NOs: 9, 10 or 11. Preferably, the transgenic plant is cold-acclimated prior to exposure to the stress condition, the stress condition is a freezing temperature from 0° C. to −30° C. and the period of time ranges from 2 hours to 72 hours. In an additional preferred aspect of the invention, the stress condition is lack of water and the period of time ranges from one to ten days, up to the wilting point. Preferably, the phenotype of the transgenic plant is characterized by increased stress tolerance compared to a non-transformed plant of the same species.

### BRIEF DESCRIPTION OF THE DRAWINGS

FIG. 1A illustrates the plasmid map of pABCTE01 (SEQ ID NO: 12).

FIG. 1B illustrates the map of the plasmid pABCTE03B (SEQ ID NO: 13) carrying the CBF2 gene for cold tolerance enhancement and the gene for reduction of lignin in Eucalyptus.

FIG. 2 illustrates that rdCBF2-7 Arabidopsis plants expressing AtCBF2 exhibited a 69% rate of survival after being exposed to freezing stress at −10° C. for 8 hours, compared to a 0.0% rate of survival of non-transformed Arabidopsis plants.

FIG. 3 illustrates that 8 of 9 CBF2-expressing Arabidopsis lines showed a decrease in electrolyte leakage upon exposure to freezing temperatures, when compared to wild-type Arabidopsis plants. rdCBF2-5 was the only CBF2-expressing Arabidopsis line showing an increase in electrolyte leakage under freezing temperatures.

FIG. 4 illustrates a transgenic eucalyptus plant carrying the AtCBF2 gene and a wild type eucalyptus plant after exposure to freezing stress. The plants were grown in a one-gallon pot for 20 days and acclimated in a transgenic fence area for 25 days before being exposed to freezing stress. For the freezing stress test, the pot was wrapped with a plastic bag to prevent desiccation and placed into a Precision Low Temperature Growth Chamber. The plants were exposed to a freezing temperature in a range between −2° and −6° C. for 48 hours, allowed to recover at 4° C. for 8 hours, and then transferred into the greenhouse. The number of plants surviving the freezing stress was scored after 5 days in the greenhouse. The photograph was taken 20 days after returning to the transgenic fence area.

FIG. 5 illustrates the results obtained from electrolyte leakage assays performed on leaves of Arabidopsis plants after exposure to freezing stress. Leaves from transgenic Arabidopsis plants expressing pd35SegCBF1 showed a decrease in electrolyte leakage compared to the leaves of non-transformed Arabidopsis plants.

FIG. 6A illustrates the plasmid map of pAGW14 containing the Eucalyptus dunnii promoter (SEQ ID NO: 9).

FIG. 6B illustrates the plasmid map of pAGW15 containing the Eucalyptus macarthurrii dehydrin promoter (SEQ ID NO 10).

FIG. 7 illustrates the induction of the Eucalyptus dunnii promoter (SEQ ID NO: 9) or the Eucalyptus macarthurrii dehydrin promoter (SEQ ID NO 10) in transgenic Arabidopsis plants exposed to low temperature (4° C.) for 24 hours.

FIG. 8 illustrates the plasmid map of pSrc-GUS (SEQ ID NO: 11).

FIG. 9 illustrates the fold induction of the Eucalyptus dunnii dehydrin promoter in two transgenic lines transformed with pAGW16 (SEQ ID NO: 18) and the fold induction of the Eucalyptus macarthurii dehydrin promoter in two transgenic lines transformed with pAGW17(SEQ ID NO: 19).

FIG. 10 illustrates the induction of the Src2 promoter (SEQ ID NO: 11) in transgenic Arabidopsis plants exposed to low temperature (4° C.) for 24 hours.

FIG. 11 illustrates the maps of four plasmids carrying promoters driving the expression of the CBF2 gene or CBF homologous genes. FIG. 11A shows the plasmid pAGSM23 carrying the Eucalyptus dunnii promoter (SEQ ID NO: 9) driving the expression of CBF homologous gene Eucalyptus gunnii CBF1 (SEQ ID NO: 5). FIG. 11B shows the plasmid pAGSM24 carrying the Eucalyptus dunnii promoter (SEQ ID NO: 9) driving the expression of CBF homologous gene Eucalyptus dunnii 8.1 (SEQ ID NO: 3). FIG. 11C shows the plasmid pAGSM42 carrying the Src promoter (SEQ ID NO: 11) driving the expression of CBF homologous gene Eucalyptus gunnii CBF1 (SEQ ID NO: 5). FIG. 11D shows the plasmid pAGSM47 carrying the Src promoter (SEQ ID NO: 11) driving the expression of the Arabidopsis thaliana CBF2 gene.

FIG. 12A illustrates the map of the plasmid pAGW16 (SEQ ID NO: 18).

FIG. 12B illustrates the map of the plasmid pAGW17 (SEQ ID NO: 19).

FIG. 13 represents a DNA gel showing an increase of the expression of Eucalyptus CBF homologues edC7.1 and edC8.1 in response to exposure to low temperatures. Three potted IPB1 plants were grown in 1-gallon pots until about two feet tall and exposed to low temperature (4° C.) for 0.5 hours, 1 hour, 2 hours, or 4 hours. At each time point, young leaves were sampled and immediately processed for total RNA extraction. A leaf sample was also taken from the plants before exposure to cold, and it was designated as sample at time zero. Poly(A) RNAs and corresponding single-strand cDNAs (sscDNA) were synthesized from total RNAs prepared from each leaf sample. 10 ng of sscDNA and 10 pmoles of the two gene-specific primers were used in a 25 μl PCR reaction. After PCR, 10 μl of each reaction were run on the DNA gels.

FIG. 14 illustrates Eucalyptus IPB1 young plants from the transgenic lines TUH000427 and TUH000435 grown in two separate 1-gallon pots prior to being exposed to water stress. Each pot contained one transgenic plant and one wild-type (WT) plant.

FIG. 15 illustrates the degree of wilting in Eucalyptus IPB1 young plants from the transgenic lines TUH000427 and TUH000435 grown in two separate 1-gallon pots and WT plants that were not watered for 8 days. Each pot contained one transgenic plant and one wild-type (WT) plant.

FIG. 16 illustrates the status of recovery of Eucalyptus IPB1 young plants from the transgenic lines TUH000427 and TUH000435 grown in two separate 1-gallon pots and WT plants at the end of a 10-day period, during which the plants were regularly watered, following an eight day-period during which the plants were not watered. Each pot contained one transgenic plant and one wild-type (WT) plant.

FIG. 17 illustrates Eucalyptus IPB1 young plants from the transgenic lines TUH000427 and TUH000435 grown in two separate 1-gallon pots prior to being exposed to water stress. Each pot contained one transgenic plant and one wild-type (WT) plant.

FIG. 18 illustrates the degree of wilting in Eucalyptus IPB1 young plants from the transgenic lines TUH000427 and TUH000435 grown in two separate 1-gallon pots and WT plants that were not watered for 8 days. Each pot contained one transgenic plant and one wild-type (WT) plant.

FIG. 19 illustrates the status of recovery of Eucalyptus IPB1 young plants from the transgenic lines TUH000427 and TUH000435 grown in two separate 1-gallon pots and WT plants at the end of a 10-day period, during which the plants were regularly watered, following an eight day-period during which the plants were not watered. Each pot contained one transgenic plant and one wild-type (WT) plant.

### DETAILED DESCRIPTION OF THE PREFERRED EMBODIMENTS

The present invention relates to processes for the genetic manipulation of stress tolerance in plants, and to transgenic plants exhibiting increased stress tolerance.

Plants vary greatly in their abilities to survive freezing temperatures and water stress. Most of the plants that originate from tropical regions have very low tolerance to freezing temperatures and limited ability to resist extreme water stress, while many species of herbaceous plants from temperate regions can survive water stress and freezing at temperatures ranging from −5° to −30° C., depending on the species. Cold tolerance may be induced by exposing the plants to low temperatures below approximately 10° C., a phenomenon known as “cold acclimation” (Hughes and Dunn, 1996; Thomashow, 1999).

Improvement of stress tolerance in economically important plants has significant practical applications, as water stress and freezing temperatures are major factors limiting the geographical locations suitable for growing crop and horticultural plants and periodically account for significant losses in plant productivity.

In some cases, conditioning of plants by exposure to several cycles of water stress renders decreases plant sensitivity to water deficits. Plants respond to acclimation by activating multiple biochemical mechanisms that lead to an increase in stress tolerance. Exposure of Arabidopsis plants to low temperature results in the rapid induction of a small family of genes encoding transcriptional activators known as C-repeat (CRT)-binding factors (CBF1, CBF2 and CBF3) or dehydration responsive element (DRE)-binding factors (DREB1b, DREB1c, and DREB1a). CBF expression activates cold-regulated genes (COR), which in turn initiate a cascade of multiple events leading to an increase in cold tolerance.

Expression of CBF genes in transgenic plants has been shown to improve tolerance to drought, high-salt and low-temperature stresses, but it is also accompanied by undesirable effects, such as growth retardation and dwarfism under normal growth conditions (Ito et al. Plant and Cell Physiology 47(1): 141-153 (2006); Lee et al. Plant, Cell & Environment 26(7): 1181-90 (2003)).

The previous work on the expression of CBF genes in transgenic plants could not presage the present inventors' discovery that CBF genes and CBF homologous genes may be expressed in transgenic plants while reducing the undesirable effects associated with CBF expression.

Accordingly, methods are provided for improving stress tolerance in plants, plant cells and plant tissues. Pursuant to this aspect of the invention, plant cells and whole plants are transformed with CBF or CBF homologous genes, which, when expressed in plant cells or in whole plants, cause an increase in stress tolerance without causing any of the previously reported undesirable effects associated with CBF expression. In a preferred embodiment, the plants or plant cells transformed with CBF or CBF homologous genes are angiosperm plants. Preferably, the plants or plant cells transformed with CBF or CBF homologous genes are Eucalyptus plants.

All technical terms used herein are terms commonly used in biochemistry, molecular biology and agriculture, and can be understood by one of ordinary skill in the art to which this invention belongs. Technical terms can be found in: Molecular Cloning: A Laboratory Manual, 3rd ed., vol. 1-3, ed. Sambrook and Russell, Cold Spring Harbor Laboratory Press, Cold Spring Harbor, N.Y., 2001; Current Protocols in Molecular Biology, ed. Ausubel et al., Greene Publishing Associates and Wiley-Interscience, New York, 1988 (with periodic updates); Short Protocols in Molecular Biology: A Compendium of Methods from Current Protocols in Molecular Biology, 5th ed., vol. 1-2, ed. Ausubel et al., John Wiley & Sons, Inc., 2002; Genome Analysis: A Laboratory Manual, vol. 1-2, ed. Green et al., Cold Spring Harbor Laboratory Press, Cold Spring Harbor, N.Y., 1997. Methodology involving plant biology techniques is described herein and is described in detail in treatises such as Methods in Plant Molecular Biology: A Laboratory Course Manual, ed. Maliga et al., Cold Spring Harbor Laboratory Press, Cold Spring Harbor, N.Y., 1995. Various techniques using PCR are described in Innis et al., PCR Protocols: A Guide to Methods and Applications, Academic Press, San Diego, 1990 and in Dieffenbach and Dveksler, PCR Primer: A Laboratory Manual, 2nd ed., Cold Spring Harbor Laboratory Press, Cold Spring Harbor, N.Y., 2003. PCR-primer pairs can be derived from known sequences by known techniques such as using computer programs intended for that purpose, Primer, Version 0.5, 1991, Whitehead Institute for Biomedical Research, Cambridge, Mass. Methods for chemical synthesis of nucleic acids are discussed, for example, in Beaucage and Caruthers, 1981, Tetra. Letts. 22: 1859-1862, and Matteucci and Caruthers, 1981 J. Am. Chem. Soc. 103: 3185.

Restriction enzyme digestions, phosphorylations, ligations and transformations were done as described in Sambrook et al., Molecular Cloning: A Laboratory Manual, 2nd ed. (1989), Cold Spring Harbor Laboratory Press. All reagents and materials used for the growth and maintenance of bacterial cells were obtained from Aldrich Chemicals (Milwaukee, Wis.), DIFCO Laboratories (Detroit, Mich.), Invitrogen (Gaithersburg, Md.), or Sigma Chemical Company (St. Louis, Mo.), unless otherwise specified.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein. It is therefore understood that modifications in the DNA sequence encoding transcription factors which do not substantially affect the functional properties of the protein are contemplated.

The term “expression” denotes the production of the protein product encoded by a gene. The term “over-expression” refers to the production of a gene product in transgenic organisms that exceeds levels of production in normal or non-transformed organisms.

### C-Repeat Binding Factor Sequences

The phrase “CBF gene” refers to a gene that encodes a transcriptional activator that binds to the CRT (C-repeat)/DRE (dehydration responsive element) DNA regulatory element present in the promoters of many cold- and drought-inducible genes, including those designated COR (cold-regulated). The phrase “homologous CBF gene” refers to a gene that shares a high sequence identity or similarity with the CBF gene and has CBF function.

In this description, the phrases “CBF gene sequence” and “CBF homologous gene sequence” denote any nucleic acid, gene, polynucleotide, DNA, RNA, mRNA, or cDNA molecule that confers stress-related plant C-repeat binding factor (CBF) activity. Illustrative of this category are polynucleotides that comprise a sequence represented by SEQ ID Nos: 1, 3, 5 or 7, that encode a polypeptide displaying CBF activity.

A CBF or homologous CBF polynucleotide sequence suitable for the present invention may be identified from a myriad of plants characterized by the presence of a CBF gene. Although the aforementioned nucleotide sequences are disclosed herein, they are not to be taken as limitations on the present invention. A CBF DNA sequence may be isolated as cDNA or genomic DNA from any suitable plant species using oligonucleotide primers or probes based on DNA or protein sequences disclosed herein. Specific examples of plant species from which CBF genes may be isolated include dicotyledons, such as Cucurbitaceae, Solanaceae, Brassicaceae, Rutaceae, Papilionaceae, such as alfalfa and Vigna unguiculata, Malvaceae, Asteraceae, Malpighiaceae such as Populus, Myrtaceae such as Eucalyptus; and monocotyledons, such as gramineae, including wheat, barley, and corn. For the purposes of the present invention, a CBF gene is preferably isolated from Arabidopsis thaliana, and a CBF homologous gene is preferably isolated from Eucalyptus.

In this description, the phrases “CBF polynucleotide sequence” and “CBF homologous polynucleotide sequence” also refer to any nucleic acid molecule with a nucleotide sequence capable of hybridizing under stringent conditions with any of the sequences disclosed herein, and coding for a polypeptide with stress-related transcription factor activity equivalent to the polypeptides comprising amino acid sequences disclosed herein under SEQ ID NOS: 2, 4,6 or 8. The phrases also include sequences which cross-hybridize with SEQ ID NO: 1, SEQ ID NO: 3, SEQ ID NO: 5, or SEQ ID NO: 7, which are at least 70% identical to the nucleotide sequence represented by SEQ ID NOs: 1, 3, 5 or 7. The nucleotide sequences of the invention may encode a polypeptide which is homologous to the polypeptides disclosed herein that comprise an amino acid sequence represented by SEQ ID NO: 2, SEQ ID NO: 4, SEQ ID NO: 6, or SEQ ID NO: 8. Further, the nucleotide sequences of the invention include those sequences that encode a polypeptide having stress-related transcription factor activity having an amino acid sequence which has at least 55%, preferably at least 60%, more preferably at least 70%, more preferably at least 80%, more preferably at least 90% and most preferably at least 95% sequence identity to an amino acid sequence disclosed herein as SEQ ID Nos: 2, 4, 6 or 8.

“Stringent conditions”, as referred to here, means conditions under which only base sequences coding for a polypeptide with stress-related transcription factor activity equivalent to the transcription factor encoded by a CBF gene sequence or CBF homologous gene sequence form hybrids with the specific CBF or CBF homologous sequence (referred to as specific hybrids), and base sequences coding for polypeptides with no such equivalent activity do not form hybrids with the specific sequence (referred to as non-specific hybrids). One with ordinary skill in the art can readily select such conditions by varying the temperature during the hybridization reaction and washing process, or the salt concentration during the hybridization reaction and washing process. Specific examples include, but are not limited to, conditions under which hybridization is brought about in 3.5×SSC, 1×Denhardt's solution, 25 mM sodium phosphate buffer (pH 7.0), 0.5% SDS, and 2 mM EDTA for 18 hours at 65° C., followed by 4 washes of the filter at 65° C. for 20 minutes, in 2×SSC, 0.1% SDS, and a final wash for up to 20 minutes in 0.5×SSC, 0.1% SDS, or 0.3×SSC and 0.1% SDS for greater stringency, and 0.1×SSC, 0.1% SDS for even greater stringency. Other conditions may be substituted, as long as the degree of stringency in equal to that provided herein, using a 0.5×SSC final wash.

Additionally, CBF homologous gene sequences include fragments and variants of the polynucleotides represented by SEQ ID Nos: 1 or 3, 5 or 7, with one or more bases deleted, substituted, inserted, or added, that code for a polypeptide with stress-related transcription factor activity. The “base sequences with one or more bases deleted, substituted, inserted, or added” referred to here are widely known by those having ordinary skill in the art to retain physiological activity even when the amino acid sequence of a protein generally having that physiological activity has one or more amino acids substituted, deleted, inserted, or added. For example, the poly A tail or 5′ or 3′ end nontranslation regions may be deleted, and bases may be deleted to the extent that amino acids are deleted. Bases may also be substituted, as long as no frame shift results. Bases also may be “added”, as long as such modifications do not result in the loss of stress-related transcription factor activity. A modified DNA in this context can be obtained by modifying the DNA base sequences of the invention so that amino acids at specific sites are substituted, deleted, inserted, or added by site-specific mutagenesis, as described in Zoller & Smith, 1982, Nucleic Acid Res. 10: 6487-6500.

### Promoters

The invention provides nucleic acid molecules that cause improved stress tolerance in a transformed plant. An important aspect of the present invention is the use of DNA constructs wherein a CBF gene or homologous CBF gene nucleotide sequence is operably linked to one or more promoters that drive the expression of the CBF gene sequence or CBF homologous gene sequence in a constitutive manner or in certain cell types, organs, or tissues, or drive the temporary expression of the CBF gene sequence or CBF homologous gene in response to exposure to stress, such as water stress and low temperatures, so as to improve stress tolerance in a transformed plant without unduly affecting its normal development or physiology.

The selected promoter should cause the expression of the CBF gene or CBF homologous gene, pursuant to the invention, to modify stress tolerance in the host plant cell, or in the host plant.

Suitable promoters are illustrated by, but are not limited to, constitutive promoters, such as the cauliflower mosaic virus CaMV 35S, maize Adh1-based pEmu, rice Actl and maize Ubi promoters; stress-responsive promoters, such as the Arabidopsis thaliana rd29A promoter; and dehydrin promoters disclosed herein, comprising a nucleic acid sequence represented by SEQ ID Nos.: 9, 10 or 11, or a fragment or variant thereof comprising a nucleic acid sequence of at least 30 contiguous nucleotides, and preferably at least 40 contiguous nucleotides, that is at least 50% identical to a dehydrin promoter having SEQ ID NOs: 9, 10 or 11. Other suitable promoters are disclosed in U.S. Pat. No. 6,380,459, U.S. patent application Ser. No. 10/702,319, U.S. patent application Ser. No. 10/717,897 and U.S. patent application Ser. No. 10/703,091, which are herein incorporated by reference.

### Plants for Genetic Engineering

The present invention comprehends the genetic manipulation of plants, to enhance their stress tolerance, by driving the expression of a CBF gene or a CBF homologous gene, preferably under the control of a promoter as described above. The result is enhanced stress tolerance.

The term “plant” denotes any fiber-containing plant material that can be genetically manipulated, including, but not limited to, differentiated or undifferentiated plant cells, protoplasts, whole plants, plant tissues, or plant organs, or any component of a plant such as a leaf, stem, root, bud, tuber, fruit, rhizome, or the like.

Plants that can be engineered in accordance with the invention include, but are not limited to, trees, such as Eucalyptus species and hybrids thereof (E. alba, E. albens, E. amplifolia, E. amygdalina, E. aromaphloia, E. baileyana, E. balladoniensis, E. benjensis, E. benthamii, E. bicostata, E. botryoides, E. brachyandra, E. brassiana, E. brevistylis, E. brockwayi, E. calmaldulensis, E. ceracea, E. cloeziana, E. coccifera, E. cordata, E. cornuta, E. corticosa, E. crebra, E. croajingolensis, E. curtisii, E. dalrympleana, E. deglupta, E. delegatensis, E. delicata, E. diversicolor, E. diversifolia, E. dives, E. dolichocarpa, E. dorrigoensis, E. dundasii, E. dunnii, E. elata, E. erythrocorys, E. erythrophloia, E. eudesmoides, E. falcata, E. gamophylla, E. glaucina, E. globulus, E. globulus subsp. bicostata, E. globulus subsp. globulus, E. gongylocarpa, E. grandis, E. grandis×urophylla, E. guilfoylei, E. gunnii, E. hallii, E. houseana, E. jacksonii, E. lansdowneana, E. latisinensis, E. leucophloia, E. leucoxylon, E. lockyeri, E. lucasii, E. macarthurii, E. maidenii, E. marginata, E. megacarpa, E. melliodora, E. michaeliana, E. microcorys, E. microtheca, E. muelleriana, E. nitens, E. nitida, E. obliqua, E. obtusiflora, E. occidentalis, E. optima, E. ovata, E. pachyphylla, E. pauciflora, E. pellita, E. perriniana, E. petiolaris, E. pilularis, E. piperita, E. platyphylla, E. polyanthemos, E. populnea, E. preissiana, E. pseudoglobulus, E. pulchella, E. radiata, E. radiata subsp. radiata, E. regnans, E. risdonii, E. robertsonii, E. rodwayi, E. rubida, E. rubiginosa, E. saligna, E. salmonophloia, E. scoparia, E. sieberi, E. spathulata, E. staeri, E. stoatei, E. tenuipes, E. tenuiramis, E. tereticornis, E. tetragona, E. tetrodonta, E. tindaliae, E. torquata, E. umbra, E. urophylla, E. vernicosa, E. viminalis, E. wandoo, E. wetarensis, E. willisii, E. willisii subsp. falciformis, E. willisii subsp. willisii, E. woodwardii); Populus species and hybrids thereof (P. alba, P. alba×P. grandidentata, P. alba×P. tremula, P. alba×P. tremula var. glandulosa, P. alba×P. tremuloides, P. balsamifera, P. balsamifera subsp. trichocarpa, P. balsamifera subsp. trichocarpa×P. deltoides, P. ciliata, P. deltoides, P. euphratica, P. euramericana, P. kitakamiensis, P. lasiocarpa, P. laurifolia, P. maximowiczii, P. maximowiczii×P. balsamifera subsp. trichocarpa, P. nigra, P. sieboldii×P. grandidentata, P. suaveolens, P. szechuanica, P. tomentosa, P. tremula, P. tremula×P. tremuloides, P. tremuloides, P. wilsonii, P. canadensis, P. yunnanensis); Conifers such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); cedars such as Western red cedar (Thuja plicata) and Alaska yellow-cedar (Chamaecyparis nootkatensis); citrus species, including C. medica, C. aurantifolia, C. latipes, C. limon; C. reticulata, C. sinensis, C. paradisi, C. aurantium, C. jambhiri, C. grandis, C. indica, C. ichangensis, C. tachibana, C. micrantha, and citrus hybrids, including, Palestine sweet lime, bergamot and Volkamer lemon, Rangpur lime and Rough lemon; avocado (Persea americana Mill), papaya (Carica papaya), nutmeg (Myristica insipida), pistachio (Pistacio vera), kiwi (Actinidia deliciosa A. Chev.), and jojoba (Simmondsia chinensis).

Fiber-producing plants also are included in this context. Illustrative crops are cotton (Gossipium spp.), flax (Linum usitatissimum), stinging nettle (Urtica dioica), hop (Humulus lupulus), lime trees (Tilia cordata, T. x. europaea and T. platyphyllus), spanish broom (Spartium junceum), ramie (Boehmeria nivea), paper mulberry (Broussonetya papyrifera), New Zealand flax (Phormium tenax), dogbane (Apocynum cannabinum), Iris species (I. douglasiana, I. macrosiphon and I. purdyi), milkweeds (Asclepia species), pineapple and banana.

The phrase “transgenic plant” refers to a plant that has incorporated a DNA sequence, including, but not limited, to genes that are not normally present in a host plant genome, DNA sequences not normally transcribed into RNA or translated into a protein (“expressed”), or any other genes or DNA sequences normally present in the non-transformed plant, that are genetically engineered or have altered expression. The phrase “transgenic plant” encompasses primary transformants regenerated from calluses obtained from transformed plant cells (R₀ plants), as well as their seed-derived R₁ and R₂ progenies, and vegetatively-propagated derivatives of the R₀ plants and R₁ and R₂ progenies. The invention also contemplates production of hybrids using an R₀, R₁ or R₂ plant as a parent.

It is contemplated that, in some instances, the genome of an inventive transgenic plant will have been augmented through the stable introduction of a transgene. In other instances, however, the introduced gene will replace an endogenous sequence. A preferred gene in the regard, pursuant to the present invention, is a CBF gene or a CBF homologous gene.

### DNA Constructs

In accordance with one aspect of the invention, a CBF gene or CBF homologous gene sequence is incorporated into a DNA construct that is suitable for plant transformation. Such a DNA construct can be used to modify CBF expression in plants, as described above.

Accordingly, DNA constructs are provided comprising a CBF gene sequence or CBF homologous gene sequence, under the control of a promoter, such as any of those mentioned above, so that the construct can generate RNA in a host plant cell.

Recombinant DNA constructs may be made using standard techniques. For example, the DNA sequence for transcription may be obtained by treating a vector containing said sequence with restriction enzymes to cut out the appropriate segment. The DNA sequence for transcription may also be generated by annealing and ligating synthetic oligonucleotides or by using synthetic oligonucleotides in a polymerase chain reaction (PCR) to give suitable restriction sites at each end. The DNA sequence then is cloned into a vector containing upstream promoter and downstream terminator sequences.

The expression vectors of the invention may also contain termination sequences, which are positioned downstream of the nucleic acid molecules of the invention, such that transcription of mRNA is terminated, and polyA sequences added. Exemplary of such terminators are the cauliflower mosaic virus CaMV 35S terminator and the nopaline synthase gene Tnos terminator. The expression vector may also contain enhancers, start codons, splicing signal sequences, and targeting sequences.

Expression vectors of the invention may also contain a selection marker by which transformed plant cells can be identified in culture. The marker may be associated with the heterologous nucleic acid molecule, i.e., the gene operably linked to a promoter. As used herein, the term “marker” refers to a gene encoding a trait or a phenotype that permits the selection of, or the screening for, a plant or plant cell containing the marker. Usually, the marker gene will encode antibiotic or herbicide resistance. This allows for selection of transformed cells from among cells that are not transformed or transfected.

Examples of suitable selectable markers include adenosine deaminase, dihydrofolate reductase, hygromycin-B-phosphotransferase, thymidne kinase, xanthine-guanine phospho-ribosyltransferase, glyphosate and glufosinate resistance and amino-glycoside 3′-O-phosphotranserase (kanamycin, neomycin and G418 resistance). These markers include resistance to G418, hygromycin, bleomycin, kanamycin, and gentamicin. The construct may also contain the selectable marker gene Bar that confers resistance to herbicidal phosphinothricin analogs like ammonium gluphosinate (Thompson et al., 1987, EMBO J. 9: 2519-2523). Other suitable selection markers are known to the person skilled in the art.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, when Agrobacterium is the host, T-DNA sequences may be included to facilitate the subsequent transfer to and incorporation into plant chromosomes.

### Plant Transformation

Constructs according to the invention may be used to transform any plant cell, using a suitable transformation technique. Both monocotyledon and dicotyledonous angiosperm or gymnosperm plant cells may be transformed in various ways known to the art. For example, see Klein et al., 1993, Biotechnology 4: 583-590; Bechtold et al., 1993, C. R. Acad. Sci. Paris 316: 1194-1199; Bent et al., 1986, Mol. Gen. Genet. 204: 383-396; Paszowski et al., 1984, EMBO J. 3: 2717-2722; Sagi et al., 1994, Plant Cell Rep. 13: 262-266.

Agrobacterium species such as A. tumefaciens and A. rhizogenes can be used, for example, in accordance with Nagel et al., 1990, Microbiol Lett 67: 325. Agrobacterium may be transformed with a plant expression vector via electroporation, followed by introduction of the Agrobacterium into plant cells via the well known leaf-disk method. Additional methods include, but are not limited to, particle gun bombardment, calcium phosphate precipitation, polyethylene glycol fusion, transfer into germinating pollen grains, direct transformation (Lorz et al., 1985, Mol. Genet. 199: 179-182), and other methods known to the art. Use of a selection marker, such as kanamycin resistance, allows quick identification of successfully transformed cells.

The Agrobacterium transformation methods discussed above are known to be useful for transforming dicots. For transformation of cereal monocots using Agrobacterium, see de la Pena et al., 1987, Nature 325: 274-276; Rhodes et al., 1988, Science 240: 204-207; and Shimamato et al., 1989, Nature 328: 274-276, all of which are incorporated by reference, have transformed. Also see Bechtold, et al., 1994, C.R. Acad. Sci. Paris 316, showing the use of vacuum infiltration for Agrobacterium-mediated transformation.

The presence of a protein, polypeptide, or nucleic acid molecule in a particular cell can be measured to determine if, for example, a cell has been successfully transformed or transfected according to methods well known in the art.

### Stress Tolerance

Transgenic plants of the invention are characterized by increased stress tolerance. The phrase “increased stress tolerance” refers to a transgenic plant that survives exposure to water stress or freezing stress and maintains its normal phenotype after survival, when compared to a wild-type or non-transformed plant of the same species that does not survive the water stress or freezing stress, or shows significant water loss or freeze damage. The terms “hardening” or “acclimatization” refer to a plant grown under conditions of suboptimal water supply. The terms “cold acclimation” or “cold acclimated” refer to a plant exposed for 5 to 25 days to a cold hardening treatment that consists in exposing the plant to a low, above-freezing temperature, while decreasing light intensity and day length. The phrase “water stress” indicates exposure of a non-hardened plant to dry conditions (lack of water) for one to ten days or up to the wilting point, followed by watering and a recovery period of 24 hours at room temperature, before transfer into the greenhouse at 22° C. The phrases “dry conditions” or “lack of water” refer to conditions that may cause incipient, temporary or permanent wilting of leaves, without causing irreversible wilting. The phrase “incipient wilting” refers to a stage of wilting of leaves that is not readily noticeable. The phrase “temporary wilting” refers to a stage of wilting which is characterized by visible drooping of the leaves during the day, from which the plant recovers at night. The phrase “permanent wilting” refers to a stage of wilting, where the plant does not recover during the overnight period. Permanently wilted plants may recover when water is added to the soil. In addition to wilting, leaves may curl or warp, become crinkly, turn brown along the edges (scorch), turn yellow, turn brown, and/or fall from the tree. The phrase “prolonged permanent wilting” refers to a stage where the plant has reached the wilting point and does not recover after addition of water. The term “wilting point” refers to the minimal point of soil moisture that the plant requires not to irreversibly wilt and indicates the limit of moisture decrease at which or under which a plant wilts and can no longer recover its turgidity when placed in a saturated atmosphere for 12 hours. Increase in water stress tolerance is assessed by scoring the number of transgenic plants surviving the water stress after 10 days in the greenhouse, compared to the number of wild-type or non-transformed plants of the same species. Increase in water stress tolerance can also be assessed by scoring the degree of wilting of shoots and leaves after exposure to water stress.

The phrase “freezing stress” indicates exposure of a cold acclimated plant to a temperature in a range between 0° and −30° C. for 2 to 72 hours, followed by a 4 to 8 hour recovery period at 4° C., before transfer into the greenhouse at 22° C. Increase in cold tolerance is assessed by scoring the number of transgenic plants surviving the freezing stress after 1 to 5 days in the greenhouse, compared to the number of wild-type or non-transformed plants of the same species. Increase in cold tolerance can also be assessed by scoring the freezing damage to leaves and shoots after exposure to freezing stress.

Specific examples are presented below of methods for obtaining DNA constructs comprising CBF genes or CBF homologous gene sequences, as well as for introducing the target genes, via Agrobacterium, to produce plant transformants. These examples are meant to be exemplary and not as limitations on the present invention.

### Example 1

### Preparation of DNA Constructs Containing the Arabidopsis CBF2 Gene

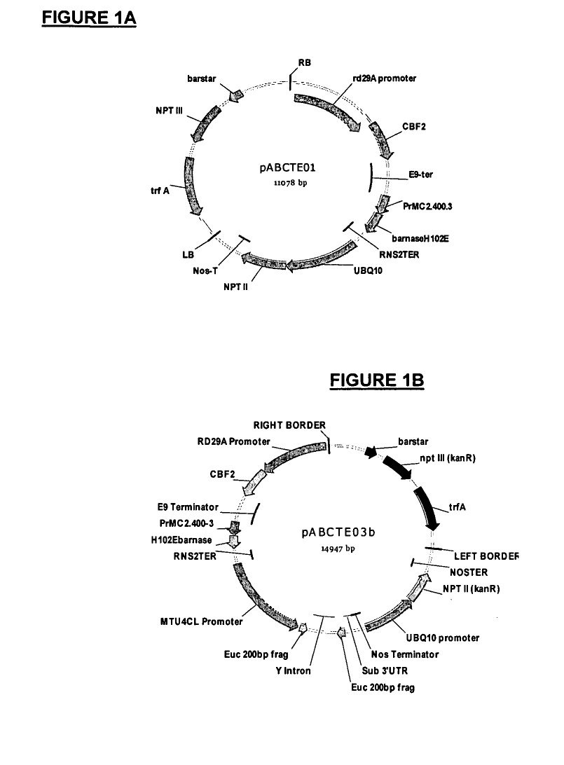

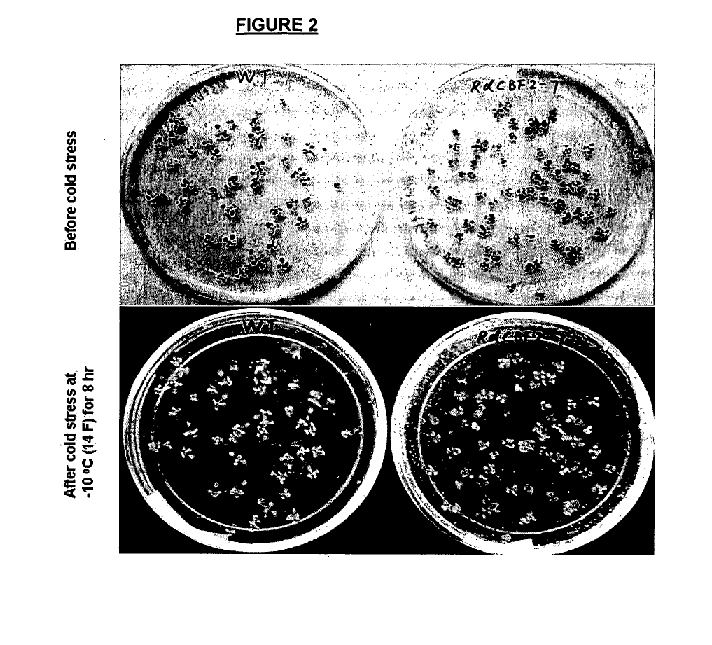

The plasmid pMEN068 containing the Arabidopsis CBF2 (AtCBF2) gene (GenBank Accession No. AF074601) and the Arabidopsis rd29A promoter driving the expression of the AtCBF2 was obtained from Mendel Biotechnology, Inc. The rd29A::CBF2::E9ter fragment in the pMEN68 plasmid was cloned into an ArborGen backbone pWVR5. The backbone vector pWVR5 is a pBI121 vector (Clontech laboratories, Palo Alto Calif.) with the 35S promoter GUS sequence removed and the NOS promoter replaced with the UBQ10 promoter from Arabidopsis (Sun, C. W & Callis, J (1997) Plant J., 11:101-111). The pABTV14 plasmid was then obtained. The rd29A::CBF2::E9ter sequence was then digested out of the pABTV14 plasmid with Kpn I//Pst I and subcloned into the pAGF243 plasmid (U.S. patent application Ser. No. 10/946,622) to obtain the pABCTE01 plasmid (FIG. 1A, SEQ ID NO: 12), which carries the ArborGen pollen control cassette PRMC2::barnaseH102E (U.S. patent application Ser. No. 10/946,622). The 4CL RNAi sequence was digested out of the pARB599 (U.S. patent application Ser. No. 11/229,856) plasmid with Not I and subcloned into the pABCTE01 plasmid (SEQ ID NO: 12) to obtain the pABCTE03B plasmid (FIG. 1B, SEQ ID NO: 13). This plasmid contained three cassettes: the rd29A::CBF2 sequence, the pollen control cassette and the 4CLRNAi cassette. The two plasmids, pABCTE01 and pABCTE03B, were used for eucalyptus transformation.

### Example 2

### Transgenic Arabidopsis Plants Expressing the CBF2 Gene have Enhanced Freezing Tolerance

Arabidopsis plants were transformed with the plasmid pMEN068 containing the rd29A promoter and the AtCBF2 gene using vacuum infiltration as described by Green P. J. Plant Physiol. 119: 331-342 (1999). T2 seeds of the transgenic Arabidopsis plants were germinated on agar containing 1×MS salt and 0.5% sucrose plus kanamycin (35 μg/ml) in Petri dishes at room temperature. No kanamycin was added into the agar where wild type control Arabidopsis seeds germinated. After 21-days of growth at 22° C. with constant lights, the new plantlets growing on the agar in the Petri dishes were incubated at 4° C. for 20 hours with light to induce the rd29A promoter, and then exposed to −10° C. for 8 hours in the dark. Following the freezing stress, the plants were transferred to the growth room (22° C.) for recovery. The number of plants surviving the freezing stress was scored after 2 days of recovery at 22° C.

Eight of nine Arabidopsis transgenic plants carrying the pMEN068 containing the rd29A::AtCBF2 sequence showed enhanced freezing tolerance. In particular, the rdCBF2-7 plant maintained its normal phenotype after freezing stress and showed the best rate of survival, with 69% of the individual plants showing freezing tolerance. In contrast, 0.0% of the wild type Arabidopsis plants survived the freezing stress. (See FIG. 2). Table 1 summarizes the results.

TABLE 1
Transgenic Arabidopsis plants carrying the
AtCBF2 gene have enhanced freezing tolerance.

| | | Total # of | # of plants | Survival |
|---|---|---|---|---|
| Plant ID | Phenotype | plants * | survived | rate (%) |
| WT | normal | 119 | 0 | 0 |
| rdCBF2-1 | normal | 44 | 20 | 45 |
| rdCBF2-3 | normal | 62 | 21 | 34 |
| rdCBF2-5 | normal | 53 | 0 | 0 |
| rdCBF2-6 | normal | 75 | 25 | 33 |
| rdCBF2-7 | normal | 65 | 45 | 69 |
| rdCBF2-9 | dwarf | 64 | 51 | 80 |
| rdCBF2-10 | normal | 71 | 31 | 44 |
| rdCBF2-11 | normal | NA | NA | NA |
| rdCBF2-12 | dwarf | 68 | 43 | 63 |
| rdCBF2-13 | semi-dwarf | 76 | 57 | 75 |

* The total # of transgenic plant was collected from a single Petri dish (150 × 100 mm) while the same # of WT was from two Petri dishes.

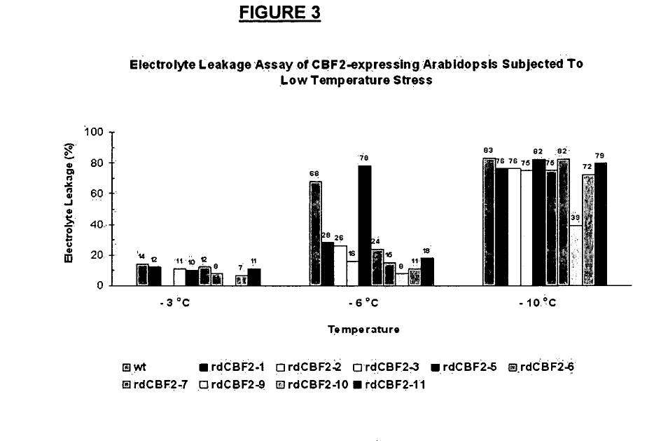

In addition, eight of nine Arabidopsis transgenic plants carrying the pMEN068 containing the rd29A::AtCBF2 sequence showed a decrease in electrolyte leakage upon exposure to freezing temperatures, when compared to wild-type Arabidopsis plants. rdCBF2-5 was the only CBF2-expressing Arabidopsis line showing an increase in electrolyte leakage under freezing temperatures (See FIG. 3).

### Example 3

### Transformation of Eucalyptus IPB1 Clones

Eucalyptus IPB1 clones were transformed with the plasmids pABCTE01 (SEQ ID NO: 12) and pABCTE03B (SEQ ID NO: 13) according to the protocol described in U.S. patent application Ser. No. 10/981,742.

### Example 4

### Transgenic Eucalyptus Plants Expressing the Cbf2 Gene have Enhanced Freezing Tolerance

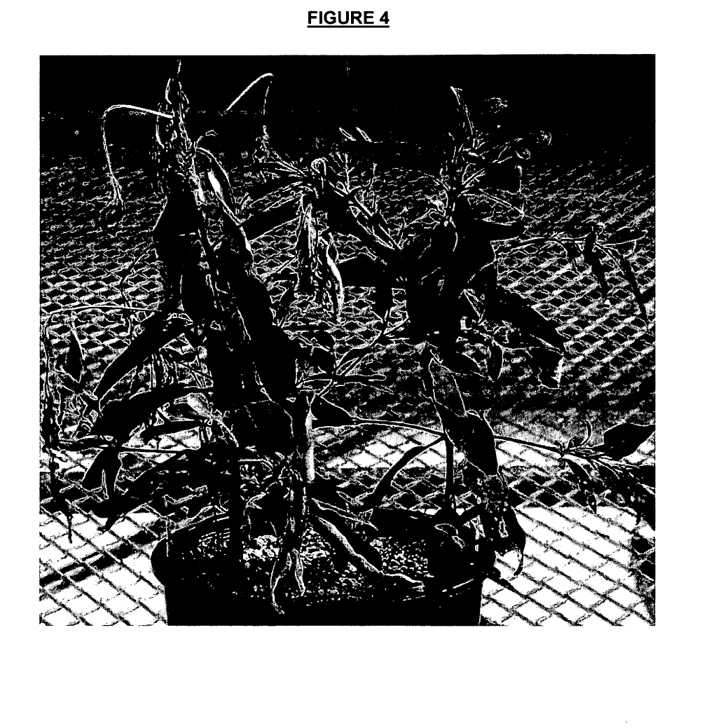

Eucalyptus plants carrying the pABCTE01 plasmid were tested for their enhanced freezing tolerance. A transgenic eucalyptus plant carrying the AtCBF2 gene and a wild type eucalyptus plant were grown in a one-gallon pot for 20 days. The plants were acclimated in a transgenic fence area for 25 days before being exposed to freezing stress. For the freezing stress test, the pot, was wrapped with a plastic bag to prevent desiccation and placed into a Precision Low Temperature Growth Chamber. The plants were exposed to a freezing temperature in a range between −2° and −6° C. for 48 hours, allowed to recover at 4° C. for 8 hours, and then transferred into the greenhouse. The number of plants surviving the freezing stress and the freezing damage of leaves and shoots were scored after 5 days in the greenhouse. FIG. 4 illustrates a transgenic Eucalyptus plant showing enhanced freezing tolerance compared to a non-transformed Eucalyptus plant.

Thirty one of 42 Eucalyptus transgenic plants carrying the pABCTE01 plasmid containing the AtCBF2 gene showed enhanced freezing tolerance under test conditions. Overall, 74% of the transgenic eucalyptus plants carrying the CBF2 gene showed enhanced freezing tolerance. In contrast, GUS or wild type eucalyptus control plants suffered significant damage. Table 2 summarizes the results of the test.

TABLE 2
Transgenic Eucalyptus plants carrying the
AtCBF2 gene have enhanced freezing tolerance.

| | Ranks of |
|---|---|
| Transgenic | freezing |
| Line No. | tolerance* |
| 1 | + |
| 2 | − |
| 3 | + |
| 4 | + |
| 5 | − |
| 6 | − |
| 7 | ++ |
| 8 | + |
| 9 | + |
| 10 | ++ |
| 11 | + |
| 12 | ++ |
| 13 | ++ |
| 14 | + |
| 15 | ++ |
| 16 | ++ |
| 17 | − |
| 18 | ++ |
| 19 | − |
| 20 | − |
| 21 | − |
| 22 | − |
| 23 | ++ |
| 24 | + |
| 25 | + |
| 26 | ++ |
| 27 | + |
| 28 | − |
| 29 | ++ |
| 30 | + |
| 31 | + |
| 32 | ++ |
| 33 | − |
| 34 | ++ |
| 35 | ++ |
| 36 | ++ |
| 37 | − |
| 38 | + |
| 39 | + |
| 40 | + |
| 41 | + |
| 42 | + |

The ranks of freezing tolerance in the transgenic eucalyptus plants are based on the results and observations recorded during the freezing stress tests. The ++ sign indicates no or slight damage during the freezing stress period. The + sign indicates significant damage during tests, but less damage than the corresponding control plant. The − sign indicates that the plant suffered as much damage as the control plant, and lacked freezing tolerance.

### Example 5

### Isolation and Identification of CBF Gene Homologues from Eucalyptus Plants

CBF homologue genes Ed7.1 (SEQ ID NO: 1) and Ed8.1 (SEQ ID NO: 3) were isolated from Eucalyptus dunnii using the yeast One-Hybrid system (Clonetech Matchmaker Yeast One-Hybrid Kit (Protocol # PT1031-1, version PR71132); Clontech, Catalog# K1603-1), with the drought-responsive element (DRE) sequence as the bait. Thirty primary clones were recovered from the system. Of these, 20 were confirmed in a 1:1 interaction with the 4×DRE reporter. Using sequence and Blast analyses, nine of the twenty clones showed significant homology to the Arabidopsis and tomato CBF transcription factors, and the Ed 7.1 and Ed 8.1 genes were identified.

The CBF homologous genes, EgCBF1 (SEQ ID NO: 5) and EgCBF2 (SEQ ID NO: 7), were isolated from Eucalyptus grandis according to the protocol used in Provisional U.S. patent Application No. 60/742,926 with the amendment that the isolated polynucleotide sequences were identified as encoding a CBF homologue based on similarity to known sequences from other plant species. The EgCBF1 cDNA isolated using this method was truncated and was missing 39 base pairs encoding 13 amino acids at the N-terminus of the EgCBF1 protein. The oligonucletides (SEQ ID NO: 14 and SEQ ID NO: 15) were synthesized and the full-length of EgCBF1 cDNA (SEQ ID NO: 5) was obtained using PCR technology. A full length EgCBF3 cDNA (SEQ ID NO: 7) was also synthesized using PCR technology and the oligonucletides having SEQ ID NO: 16 and SEQ ID NO: 17.

The PCR products were then digested with the restriction enzymes Sal I and Not I and directly cloned into pMEN203, which contained the CaMV 35S promoter (provided by Mendel Biotechnology), giving rise to the constructs of pd35SEgCBF1 and pd35SEgCBF3. These two constructs were suitable for transformation of Arabidopsis, but not for eucalyptus. The fragment of 35S::EgCBF3 carried by pd35SEgCBF3 was cut out using the restriction enzyme Pst I and the resulting fragment was cloned into pWVCZ2, a plasmid made using the pWVR5 backbone (see example 1), and adding the PRAG1 promoter (U.S. patent application Ser. No. 10/946,622) and the RNS2 cDNA obtained from Michigan Technology University, to give rise to pAB35SegCBF3, which is suitable for transformation of eucalyptus.

### Example 6

### Transformation of Eucalyptus Calmaldulensis Clones

Eucalyptus calmaldulensis clones were transformed with the plasmid pAB35SegCBF3 using the protocol described in U.S. patent application Ser. No. 09/153,320.

### Example 7

### CBF Gene Homologues from Eucalyptus Enhance Freezing Tolerance in Arabidopsis and Eucalyptus

Transgenic Arabidopsis plants transformed using the method described in Green Plant Physiol. 119: 331-342 (1999), carrying the pdS35EgCBF1 plasmid (35S::EgCBF1), were tested for their enhanced freezing tolerance. The T2 seeds were germinated on agar in Petri dishes and the plantlets were tested for freezing tolerance as described in Example 2.

Arabidopsis transgenic plants carrying the pdS35EgCBF1 plasmid showed enhanced freezing tolerance. In particular, the egcbf1-5 and egcbf1-6 plants showed the best rate of survival, with 43% of the individual plants surviving the freezing stress. In contrast, 0.0% of the wild type Arabidopsis plants survived the freezing stress. Table 3 summarizes the results.

TABLE 3
Transgenic Arabidopsis plants carrying the
EgCBF1 gene have enhanced freezing tolerance.

| | Total # of | # of Survived | |
|---|---|---|---|
| Plate ID | Plants | plants | Survival % |
| WT (a) | 84 | 0 | 0 |
| WT (b) | 113 | 0 | 0 |
| egcbf1-4 | 71 | 18 | 25 |
| egcbf1-5 | 68 | 29 | 43 |
| egcbf1-6 | 77 | 33 | 43 |
| egcbf1-7 | 66 | 7 | 11 |
| egcbf1-10 | 49 | 6 | 12 |
| egcbf1-11 | 96 | 25 | 26 |
| egcbf1-12 | 96 | 21 | 22 |

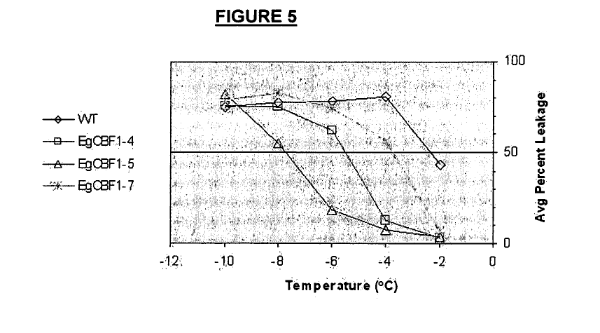

In addition, electrolyte leakage assays performed on leaves of Arabidopsis plants after exposure to freezing stress showed that leaves from transgenic Arabidopsis plants expressing pd35SegCBF1 showed a decrease in electrolyte leakage compared to the leaves of non-transformed Arabidopsis plants (See FIG. 5).

Transgenic Eucalyptus calmaldulenis plants transformed as described in Example 6 and incorporating the pAB35SegCBF3 were tested for their enhanced freezing tolerance, as described in Example 4 with some minor changes. Plants were stressed at −3.5° C. for 24 hours, and then the temperature was raised to 4° C. overnight for recovery before being transferred to greenhouse. The results were recorded 3 days after transfer to greenhouse and are shown in Table 4.

The results showed that 4 of the 6 transgenic E. calmaldulensis plants (67%) overexpressing the EgCBF3 gene had enhanced cold tolerance when compared to untransformed control plants or GUS control plants.

TABLE 4

| | | | | dieback | % of dying | |
|---|---|---|---|---|---|---|
| | ramet | construct | plant height | on top | back on | plant body |
| plant ID | # | carried | (inch) | (inch) | top | phenotypes |
| CTE520291 | 3 | pAB35SegCBF3 | 23.0 | 13.0 | 57 | normal |
| GUS | 1 | GUS | 17.0 | 10.0 | 59 | normal |
| CTE520293 | 7 | pAB35SegCBF3 | 10.0 | 0.0 | 0 | semi-dwarf |
| Control | 2 | None | 18.0 | 8.0 | 44 | normal |
| CTE520295 | 9 | pAB35SegCBF3 | 17.5 | 0.0 | 0 | normal |
| Control | 3 | None | 20.0 | 4.5 | 23 | normal |
| CTE520303 | 7 | pAB35SegCBF3 | 4.5 | 0.0 | 0 | dwarf |
| Control | 4 | None | 27.0 | 11.0 | 41 | normal |
| CTE520307 | 5 | pAB35SegCBF3 | 4.5 | 0.0 | 0 | dwarf |
| GUS | 9 | GUS | 16.5 | 3.0 | 18 | normal |
| CTE520310 | 8 | pAB35SegCBF3 | 17.5 | 6.0 | 34 | normal |
| GUS | 8 | GUS | 20.0 | 9.0 | 45 | normal |

### Example 8

### Over-Expression of the Eucalyptus CBF Gene, ed8.1, Enhances Freezing Tolerance in Transgenic Arabidopsis

T2 seed containing the plasmid pABTV20 (rd29A::ed8.1) were germinated on agar in Petri dishes and the plantlets were tested for enhanced freezing tolerance as described in Example 2. Six of 10 tested transgenic lines had better freezing tolerance when compared to the wild type (WT) as indicated by the number of surviving plants after the freezing stress. All of the non-transformed (WT) Arabidopsis plants were dead under same freezing stress condition (Table 5).

TABLE 5
Transgenic Arabidopsis plants expressing
the ed8.1 have enhanced freezing tolerance.

| | # of plant | # of plants survived | |
|---|---|---|---|
| Line ID | stressed | after the stress | survival % |
| WT | 399 | 0 | 0 |
| pABTV20-1 | 125 | 25 | 20 |
| pABTV20-6 | 183 | 68 | 37 |
| pABTV20-9 | 268 | 0 | 0 |
| pABTV20-10 | 209 | 0 | 0 |
| pABTV20-13 | 196 | 19 | 10 |
| pABTV20-17 | 170 | 38 | 22 |
| pABTV20-21 | 168 | 30 | 18 |
| pABTV20-25 | 142 | 83 | 58 |
| pABTV20-28 | 113 | 72 | 64 |
| pABTV20-29 | 139 | 7 | 5 |

### Example 9

### Over-Expression of the Eucalyptus CBF Gene, ed8.1, Enhances Freezing Tolerance in Transgenic Eucalyptus

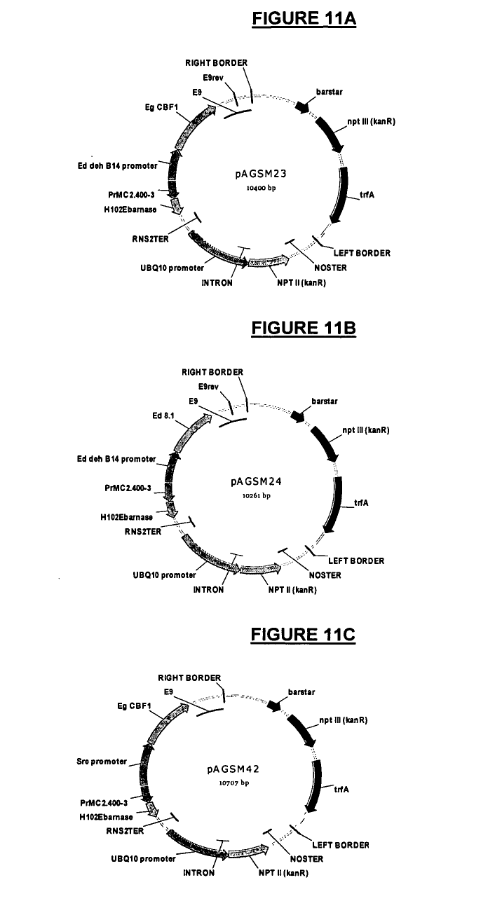

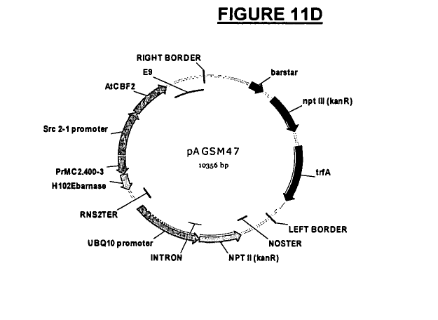

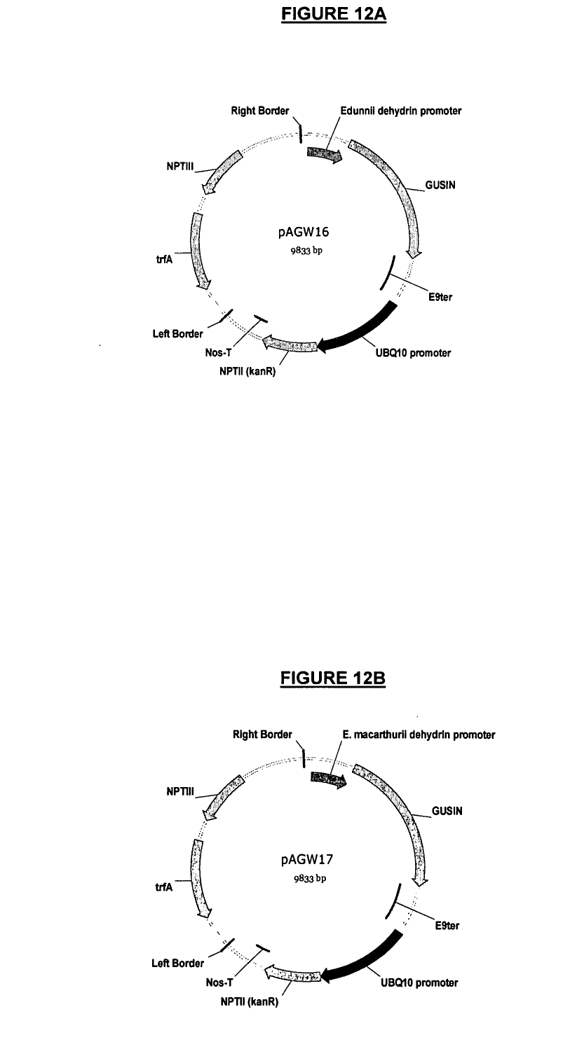

Transgenic IPB1 plants transformed as described in Example 3 and incorporating the plasmid pAGSM24 (FIG. 11B) were tested for their enhanced freezing tolerance as described in Example 4 with minor changes. The plants were stressed at −5° C. for 24 hours, transferred to 4° C. overnight, and then moved to a greenhouse. The number of plants surviving the freezing stress and the freezing damage of leaves and shoots were scored after 5 days in the greenhouse. The results showed that 4 of 13 transgenic lines (31%) tested in the chamber had enhanced freezing tolerance (Table 6).

TABLE 6
Results of chamber tests for enhanced freezing
tolerance in IPB1 plants carrying pAGSM24

| | | Enhanced Freezing |
|---|---|---|
| Transgenic Line ID | Construct | Tolerance (Yes/No) |
| TGU529930 | pAGSM24 | No |
| TGU529931 | pAGSM24 | No |
| TGU529932 | pAGSM24 | Yes |
| TGU529933 | pAGSM24 | No |
| TGU529934 | pAGSM24 | Yes |
| TGU529935 | pAGSM24 | Yes |
| TGU529936 | pAGSM24 | Yes |
| TGU529937 | pAGSM24 | No |
| TGU529938 | pAGSM24 | No |
| TGU529939 | pAGSM24 | No |
| TGU529941 | pAGSM24 | No |
| TGU529942 | pAGSM24 | No |
| TGU529943 | pAGSM24 | No |

### Example 10

### The Expression of Eucalyptus CBF Homologues, edC7.1 and edC8.1 egCBF1 and egCBF3 in Eucalyptus in Response to Cold

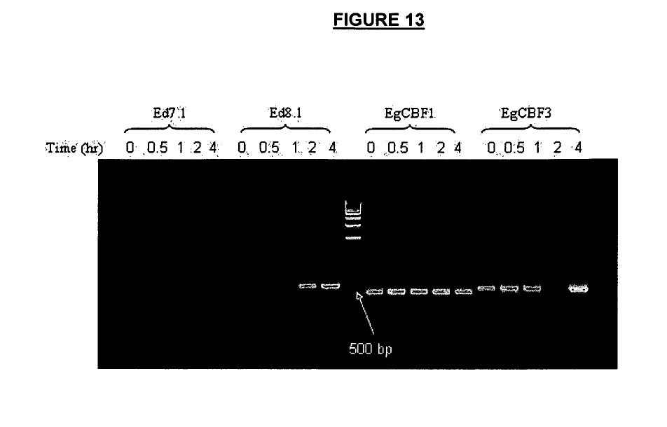

These experiments were set up to investigate the expression of the Eucalyptus CBF homologues edC7.1, edC8.1, egCBF1 and egCBF3 in Eucalyptus in response to cold. Three potted IPB1 plants were grown in 1-gallon pots until about two feet tall and then were exposed to low temperature (4° C.) for 0.5 hours, 1 hour, 2 hours, or 4 hours. At each time point, young leaves were sampled and immediately processed for total RNA extraction. A leaf sample was also taken from the plants before exposure to cold, and it was designated as sample at time zero. Poly(A) RNAs and corresponding single-strand cDNAs (sscDNA) were synthesized from total RNAs prepared from each leaf sample. 10 ng of sscDNA and 10 pmoles of the two gene-specific primers were used in a 25 μl PCR reaction. After PCR, 10 μl of each reaction were run on the DNA gels (see FIG. 13). This experiment was repeated twice. The expected sizes of the PCR products are listed below.

| | |
|---|---|
| Primers | Expected Products |

| | | |
|---|---|---|
| 1. | EdC7.1 | 439 bp |
| 2. | EdC8.l | 483 bp |
| 3. | EgCBF1 | 369 bp |
| 4. | EgCBF3 | 431 bp |

TABLE 7
Expression of Eucalyptus CBF homologues
in IPB1 plants in response to cold treatment

| | |
|---|---|
| Exposure Time | Gene Expression Levels* |

| (hours) to 4° C. | Ed7.1 | Ed8.1 | EgCBF1 | EgCBF3 |
|---|---|---|---|---|
| 0.0 | − | − | ++++ | ++++ |
| 0.5 | +/− | + | ++++ | ++++ |
| 1.0 | + | ++ | ++++ | ++++ |
| 2.0 | ++ | +++ | ++++ | na |
| 4.0 | +++ | ++++ | ++++ | ++++ |

*−, no visible PCR product on the gel; +/−, trace amount of PCR product observed on the gel; +, low level of PCR product observed on the gel; ++, medium level of PCR product observed on the gel; +++, high level of PCR product observed on the gel; ++++, very high level of PCR product observed on the gel.

The results show that the Eucalyptus CBF homologues, edC7.1 and edC8.1, are responsive to low temperature, and their expression was induced by cold. The cold induction was observed as early as 30 minutes and up to 4 hour exposure to cold. The egCBF1 and egCBF3 genes are not induced by low temperature.

### Example 11

### Isolation and Identification of Dehydrin Promoters from Eucalyptus Plants

Total RNA was extracted from the leaves of Eucalyptus dunnii and Eucalyptus macarthurii seedlings that were untreated or had been subjected to a low temperature acclimation treatment followed by exposure to freezing temperature stress. The low temperature acclimation treatment regime consisted of 8 hours of light at 11° C. and 16 hours of dark at 2.5° C. for 5 days. At the end of this 5-day period, the plants were freeze-stressed at −5° C. for 2 hours. Plant material was collected from the 4 different species/treatment combinations, snap-frozen in liquid nitrogen, and transferred to—80° C. for storage prior to RNA extraction. Total RNA was extracted from the plant material using standard techniques.

The total RNA preparations were used to generate cDNA libraries enriched for cold-inducible genes for each Eucalyptus species. Cold-inducible genes, as defined herein, are those genes that are upregulated as a result of exposure of the plants to a low temperature (4° C.). Subtracted cDNA libraries were created using the Clontech PCR-Select cDNA Subtraction Kit (Catalog #637401, Clontech Laboratories Inc, Mountain View Calif., a division of Takara Bio Inc). Comparison of cDNA libraries obtained from untreated versus low temperature-treated plants for both species of Eucalyptus plants allowed subtraction from the cDNA libraries of those genes common to both treatment groups that are not affected by exposure of the plants to the low temperature. The remaining cDNA molecules in the gene-enriched pools were then cloned into high-copy plasmids and differentially screened for cold-induced genes.

From each Eucalyptus library 100 colonies were selected and plasmid DNA isolated. Duplicate dot-blots were made from the DNA preparations. A 96-well dot-blotting apparatus was used to blot aliquots of each DNA sample to nylon membrane filters. Each DNA sample was applied onto two individual membrane filters. The DNA was fixed to the membrane filters by UV-cross-linking prior to probing the blots. For each sample, one membrane filter was probed with the cDNA library isolated from the untreated sample, and the second membrane filter was probed with the cDNA library obtained from the plants subjected to low temperature. This differential screening process allowed for the identification and isolation of genes that are cold-induced. Genes which strongly hybridized with the cDNA library obtained from the low temperature-treated plants, but did not hybridize with the cDNA library isolated from the untreated plants, were considered likely to be cold-inducible. Those genes that hybridized equally well to cDNA libraries from both low temperature-treated and untreated plants were considered as not affected by the low temperature-treatment and therefore of no interest. Genes that hybridized strongly to the cDNA library of untreated plants, but did not hybridize strongly to the cDNA library obtained from the low temperature-treated plants were considered to be down-regulated by the cold-treatment, and therefore of no interest. Approximately 10-15 clones containing putative cold-inducible genes were chosen from each species for further analysis.

The selected clones were subjected to sequence analysis, and the sequence data were used to search the Entrez database for similar sequences. The clones were found to be similar to several different genes. In particular, the sequence of some of the clones was homologous to the sequence of dehydrin genes. This was of particular interest since dehydrin genes are known to be cold-inducible. The dehydrin-homologous fragments isolated from Eucalyptus dunnii (SEQ ID NO: 9) and Eucalyptus macarthurii (SEQ ID NO: 10) showed extremely high level of identity to each other (>98%) at the nucleotide level; although they were not 100% identical. A single clone from each species was selected for further analysis.

Genome walking carried out on the dehydrin-homologous DNA clones isolated from each species led to the cloning of promoter fragments of approximately 600 bp. The DNA fragments from the two different Eucalyptus species showed a very high degree of identity to each other with only occasional single-base differences between the two. The DNA fragments also contained two conserved CBF-binding motifs, suggesting that the promoters were most likely cold-inducible.

### Example 12

### The Activity of Eucalyptus Dunnii and Eucalyptus Macarthurii Dehydrin Promoters is Induced in Transgenic Arabidopsis and Eucalyptus Plants by Exposure to Low Temperature

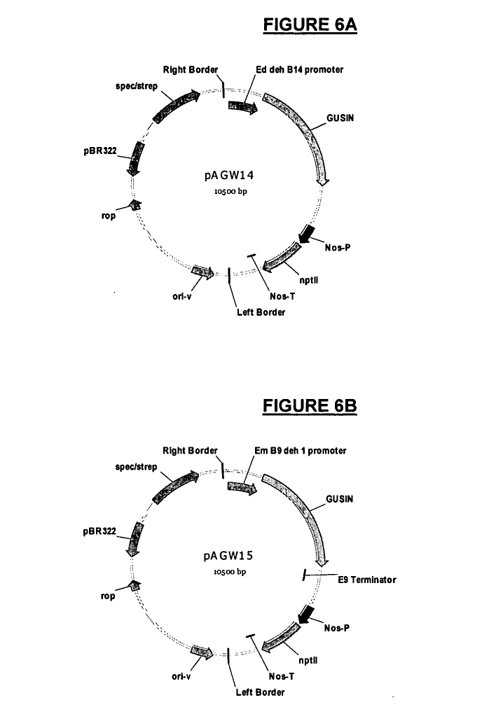

In order to study the activity of the putative dehydrin promoters, the promoter fragments obtained as described in Example 6 were cloned upstream of the GUS gene in a pBlueScript II SK(+) backbone (Stratagene, La Jolla, Calif.). These promoter:GUSIN cassettes were then cloned upstream of the E9ter in pMEN203 35S-CBF2 (obtained from Mendel; the promoter::GUSIN cassette replaced the 35S::CBF2 cassette), creating the Arabidopsis expression vectors, pAGW14 (FIG. 6A) containing the promoter isolated from Eucalyptus dunnii (SEQ ID NO: 9) and pAGW15 plasmid (FIG. 6B) containing the promoter isolated from Eucalyptus macarthurii (SEQ ID NO: 10). These vectors were transformed into Agrobacterium, and the Agrobacterium clones were used to transform Arabidopsis.

T1 plants, T1 seeds, T2 plants, and T2 seeds were generated from several lines carrying either the pAGW14 or the pAGW15 plasmid DNA. T2 seeds were germinated and the resulting seedlings were transferred to 9″×4″ pots. The plantlets thus obtained were used to study the activity of the putative dehydrin promoters.

Nine transgenic lines of pAGW14 and ten transgenic lines of pAGW15 were analyzed for the induction of the promoter activities by low temperature. Individual pots for each transgenic line were exposed to 4° C. for 24 hours. The individual plants were then cut away from the roots at the soil line, placed in a tube, snap-frozen in liquid nitrogen, and stored at −80° C. for further analysis. Plants that had not been exposed to the low temperature-treatment were also collected, stored and used as control.

GUS expression in the collected tissue samples was determined by real-time reverse-transcriptase QRT-PCR analysis; using TaqMan chemistry as per the kit manufacturer's instructions (Stratagene Brilliant QPCR Master Mix, Catalog #600549; Stratagene, La Jolla Calif.). The reactions were run on a Stratagene Mx3000P Real Time PCR machine with cycling conditions as follows: one cycle at 95° C. for 2 minutes followed by 40 cycles of (95° C., 10 seconds+58° C., 30 seconds).

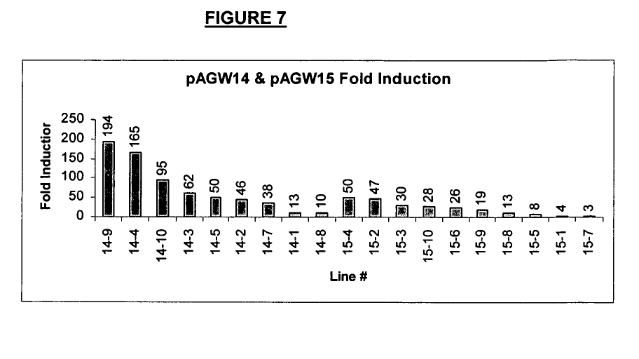

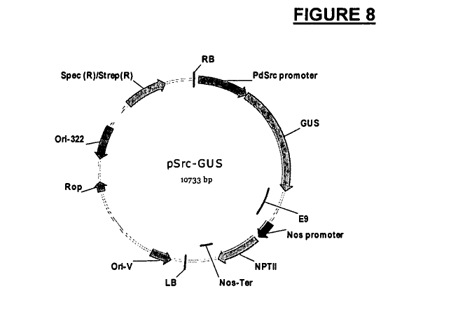

The activity of the two isolated dehydrin promoters in all transgenic lines was induced by the low temperature. In the low temperature-treated transgenic Arabidopsis plants, the activity of the dehydrin promoter isolated from Eucalyptus dunnii increased from 10 to 194 fold, and the activity of the dehydrin promoter isolated from Eucalyptus macarthurii increased from 3 to 50 fold, when compared to untreated transgenic plants. (See FIG. 7). These results clearly showed that the isolated dehydrin promoter fragments have cold-inducible activity.

The dehydrin promoters from of Eucalyptus dunnii and Eucalyptus macarthurii were also tested in Eucalyptus by transforming the plasmids pAGW16 (SEQ ID NO: 18) and pAGW17 (SEQ ID NO: 19) into Eucalyptus calmaldulensis using the protocol described in Example 6. pAGW16 and pAGW17 were created by cloning the dehydrin promoter fragments upstream of GUSIN::E9ter in pABTV16, thus replacing the rd29A promoter of the rd29A::GUSin cassette with a dehydrin promoter.

Two lines each for E. camaldulensis plants carrying either pAGW16 or pAGW17 were subjected to acclimation conditions for 5 days comprising 9 hours at 18° C. with light followed by 15 hours at 4° C. without light.

Leaf samples were collected for each line immediately prior to initiation of the cold-acclimation treatment as well as at the end of the 5-day experimental period. RNA was isolated from leaves using standard techniques. GUS expression levels were analyzed by QPCR using TaqMan chemistry as described above.

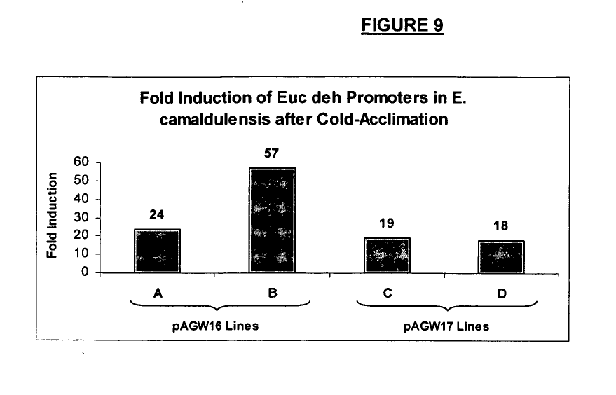

For each transgenic line, the basal level of GUS expression (i.e., the level of GUS expression in the non-cold treated samples) was assigned a value of 1, and the fold-induction for that line's cold-treated samples were calculated relative to 1. The two lines tested that were transformed with pAGW16 (SEQ ID NO: 18) had a 24 and 57 fold induction of the Eucalyptus dunnii dehydrin promoter and the two lines that were transformed with pAGW17 (SEQ ID NO: 19) had a 19 and 18 fold induction of the Eucalyptus macarthurii dehydrin promoter (See FIG. 9).

### Example 13

### The Activity of Populus Deltoids Src2 Promoter is Induced by Exposure to Low Temperature in Transgenic Arabidopsis

A SRC2 homologue promoter sequence PdSrc (SEQ ID NO: 11) was isolated from cottonwood (Populus deltoids), clone WV94, by PCR and fused with the GUS sequence. The resulting cassette, PdSrc::GUS plasmid DNA, was subcloned to obtain the pSrc-GUS vector. This vector was used for Arabidopsis transformation.

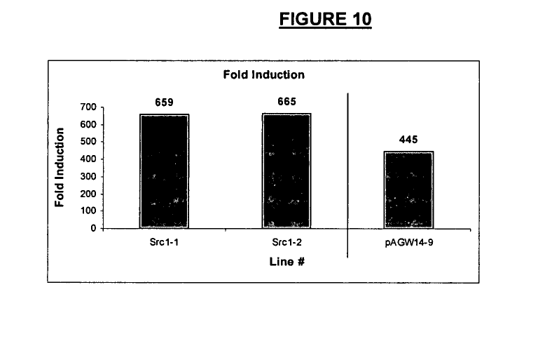

Two Arabidopsis transgenic lines containing the pSrc-GUS vector were exposed to a low temperature (4° C.) for 24 hours. The activity of the Src2 promoter was enhanced by exposure of the transgenic plants to the low temperature (See FIG. 10)

### Example 14

### Transgenic Eucalyptus Plants Carrying the AtCBF2 Gene Show Enhanced Cold Tolerance in the Field

Forty-four transgenic Eucalyptus IPB1 line plants carrying the AtCBF2 gene (pABCTE01), two IPB1 line plants carrying the GUS vector and several non-transgenic IPB1 plants were planted in the field in Loxley, Ala., in November 2005. Eight replicas for each transgenic and non-transgenic line were included in the test.

Damage caused by the exposure to a freezing temperature was assessed twice during the 2005-2006 winter season. In the first survey performed on Dec. 14, 2005, all plants, transgenic and non-transgenic, looked well-established and had grown new shoots and leaves. The height of the plants ranged from 7 to 15 inches. None of the plants showed any sign of cold damage, since the plants had not been exposed to a low temperature yet.

Between Nov. 15, 2005 and Feb. 15, 2006, the thermometer in the testing field recorded 21 freezing events during which the ambient temperature was equal to or lower than 0° C. (32° F.). The longest freezing event lasted 11 hours and the shortest freezing event lasted 0.25 hours. During three of these events the ambient temperature was also recorded to be below or equal to −3.89° C. (25° F.) but above −6.67° C. (20° F.) for a total of 10.25 hours, the longest lasted 5 hours and the shortest lasted 1 hour. Overall, there were 113.75 cumulative freezing hours during the 21 freezing events. After Feb. 15, 2006, no freezing events were recorded. Between Nov. 15, 2005 and Feb. 15, 2006, according to the website of Weather Underground, there were 52 days with average temperature below 12° C. (53.6° F.) at Mobil Airport in Alabama. The airport is located about 15 miles west from the field test site. Temperatures between 0° and 12° C. (32.0° and)53.6° impose chilling stress, but not freezing stress.

In the second survey performed on Mar. 3, 2006, all non-transgenic plants and some of the IPB1 plants carrying the AtCBF2 gene carried dieback, a dead portion on the top of a primary stem. Table 8 below summarizes the results obtained from cold damage survey on Mar. 3, 2006.

TABLE 8

| | | | Statistical Significance of | |
|---|---|---|---|---|
| Transgenic | Construct | Mean of Dieback | Dieback against IPB1** | |
| Line ID | Carried | Percentage (%)* | ρ ≦ 0.01 | ρ ≦ 0.001 |
| IPB1 | none | 21.1 | na | na |
| TUG000287 | GUS | 23.1 | na | na |
| TUG000294 | GUS | 20.2 | No | No |
| TUG000419 | AtCBF2 | 11.8 | No | No |
| TUG000424 | AtCBF2 | 36.1 | na | na |
| TUG000426 | AtCBF2 | 1.0 | Yes | Yes |
| TUG000427 | AtCBF2 | 0.5 | Yes | Yes |
| TUG000428 | AtCBF2 | 8.3 | Yes | No |
| TUG000429 | AtCBF2 | 2.1 | Yes | Yes |
| TUG000430 | AtCBF2 | 75.0 | na | na |
| TUG000431 | AtCBF2 | 1.4 | Yes | Yes |
| TUG000432 | AtCBF2 | 2.5 | Yes | Yes |
| TUG000434 | AtCBF2 | 0.5 | Yes | Yes |
| TUG000435 | AtCBF2 | 0.9 | Yes | Yes |
| TUG000436 | AtCBF2 | 42.2 | na | na |
| TUG000439 | AtCBF2 | 88.1 | na | na |
| TUG000440 | AtCBF2 | 0.0 | Yes | Yes |
| TUG000441 | AtCBF2 | 32.1 | na | na |
| TUG000442 | AtCBF2 | 18.8 | No | No |
| TUG000447 | AtCBF2 | 1.4 | Yes | Yes |
| TUG000448 | AtCBF2 | 13.6 | No | No |
| TUG000449 | AtCBF2 | 0.8 | Yes | Yes |
| TUG000450 | AtCBF2 | 3.7 | Yes | Yes |
| TUG000451 | AtCBF2 | 16.2 | No | No |
| TUG000452 | AtCBF2 | 5.9 | Yes | Yes |
| TUG000453 | AtCBF2 | 5.3 | Yes | Yes |
| TUG000454 | AtCBF2 | 18.3 | No | No |
| TUG000455 | AtCBF2 | 3.7 | Yes | Yes |
| TUG000456 | AtCBF2 | 1.5 | Yes | Yes |
| TUG000458 | AtCBF2 | 100 | na | na |
| TUG000459 | AtCBF2 | 28.8 | na | na |
| TUG000470 | AtCBF2 | 0.4 | Yes | Yes |
| TUG000472 | AtCBF2 | 10.4 | Yes | No |
| TUG000482 | AtCBF2 | 17.3 | No | No |
| TUG000484 | AtCBF2 | 3.3 | Yes | Yes |
| TUG000486 | AtCBF2 | 100 | na | na |
| TUG000489 | AtCBF2 | 0.5 | Yes | Yes |
| TUG000490 | AtCBF2 | 3.2 | Yes | Yes |
| TUG000491 | AtCBF2 | 0.7 | Yes | Yes |
| TUG000493 | AtCBF2 | 1.4 | Yes | Yes |
| TUG000494 | AtCBF2 | 0.0 | Yes | Yes |
| TUG000495 | AtCBF2 | 5.2 | Yes | No |
| TUG000512 | AtCBF2 | 8.3 | No | No |
| TUG000517 | AtCBF2 | 5.4 | Yes | Yes |
| TUG000518 | AtCBF2 | 0.0 | Yes | Yes |
| TUG000522 | AtCBF2 | 0.0 | Yes | Yes |
| TUG000523 | AtCBF2 | 4.6 | Yes | Yes |

*The dieback percentage referred to the percentage of dead portion on the top of a primary stem in the plant against the total height from soil to shoot tip of the plant. The mean of the dieback percentage was the average of eight replicas. A value of 100 for the mean of dieback percentage for a certain plant line means that all eight plants of the transgenic line were dead at the time of survey.
**The comparison of mean of dieback percentage between the non-transgenic IPB1 line and the transgenic IPB1 line was statistically analyzed using the t-test for paired two-sample for means. If one replica was absent in a sample, the analysis was done by the t-test for two-sample assuming equal variances. If the value of mean of dieback percentage of a transgenic plant line was greater than the value of the non-transgenic IPB1 line, the t-test was not applied and the sample was marked as “na” (not applied).

The results obtained from the field test during the 2005-2006 winter season strongly suggest that the Eucalyptus IPB1 line plants carrying the AtCBF2 gene have enhanced tolerance when exposed to chilling and/or freezing temperature. Under the winter conditions described above, 66% of the Eucalyptus IPB1 line plants carrying the AtCBF2 gene (29 of 44) showed a highly significant (p≦0.01) reduction of dieback of the primary shoot when compared to the non-transgenic IPB1 line, and 59% (26 of 44) showed a very highly significant (p≦0.001) reduction in dieback. Some Eucalyptus IPB1 line plants carrying the AtCBF2 gene were completely free of dieback, whereas 100% of the non-transgenic IPB1 plants carried dieback in the field.

Five of the above lines were again analyzed after another 21 months. From March 2006 to December 2007, the thermometer in the testing field recorded 37 freezing events during which the ambient temperature was equal to or lower than 0° C. (32° F.). The longest freezing event lasted 14.75 hours and the shortest freezing event lasted 0.25 hours. During five of these events the ambient temperature was also recorded to be below or equal to −3.89° C. (25° F.) but above −6.67° C. (20° F.) for a total of 28 hours, the longest lasted 10.75 hours and the shortest lasted 2.25 hours. Overall, there were 197 cumulative freezing hours during the 37 freezing events.

Table 9 below summarizes survival, height and growth data of the five transgenic lines and control lines that were assessed in December 2007.

TABLE 9

| | | | | | | Net | | Net Volume |
|---|---|---|---|---|---|---|---|---|
| | | | | Volume | | Height | DBH | Index |
| Line | | Height | DBH | Index | Relative | Growth | Growth | Growth |
| # | Survival | (Ft) | (In) | (Ft\hat{ }3) | Volume | (Ft) 2007 | (In) 2007 | (Ft\hat{ }3) 2007 |
| IPB1 | 97% | 38.2′ | 4.14″ | 4.607 | 89% | 18.4′ | 2.07″ | 3.994 |
| #427 | 100% | 37.1′ | 4.02″ | 4.190 | 81% | 18.0′ | 2.01″ | 3.649 |
| #435 | 100% | 35.7′ | 3.63″ | 3.300 | 64% | 18.4′ | 1.62″ | 2.804 |
| #470 | 100% | 34.6′ | 3.22″ | 2.505 | 48% | 17.9′ | 1.38″ | 2.105 |
| #490 | 100% | 36.1′ | 4.52″ | 5.171 | 100% | 17.9′ | 2.36″ | 4.574 |
| #494 | 87% | 37.0′ | 3.46″ | 3.109 | 60% | 18.8′ | 1.56″ | 2.648 |

The five above lines were planted in another two plots at the same site in Loxley, Ala., in July and August 2006 and analyzed for survival and growth in December 2007. The freezing events recorded are the same as those listed above for the period of March 2006 to December 2007 at Loxley, Ala. as no freezing events were recorded between March and August.

Table 10 (planted July 2006) & Table 11 (planted August 2006) below summarize survival, height and growth data of the transgenic lines and control lines that were assessed in December 2007.

TABLE 10

| | | | | | | Net | | Net Volume |
|---|---|---|---|---|---|---|---|---|
| | | | | Volume | | Height | DBH | Index |
| Line | | Height | DBH | Index | Relative | Growth | Growth | Growth |
| # | Survival | (Ft) | (In) | (Ft\hat{ }3) | Volume | (Ft) 2007 | (In) 2007 | (Ft\hat{ }3) 2007 |
| IPB1 | 100% | 29.9′ | 3.48″ | 2.539 | 100% | 23.6′ | 3.01″ | 2.373 |
| #427 | 100% | 28.4′ | 2.86″ | 1.638 | 64% | 19.9′ | 2.26″ | 1.616 |
| #435 | 100% | 27.8′ | 2.82″ | 1.569 | 62% | 19.3′ | 2.21″ | 1.546 |
| #470 | 100% | 25.9′ | 2.44″ | 1.088 | 43% | 18.3′ | 1.93″ | 1.073 |
| #490 | 100% | 29.4′ | 3.31″ | 2.286 | 90% | 20.8′ | 2.66″ | 2.259 |
| #494 | 100% | 26.5′ | 2.81″ | 1.492 | 59% | 17.7′ | 2.13″ | 1.463 |

TABLE 11

| | | | | | | Net | | Net Volume |
|---|---|---|---|---|---|---|---|---|
| | | | | Volume | | Height | DBH | Index |
| Line | | Height | DBH | Index | Relative | Growth | Growth | Growth |
| # | Survival | (Ft) | (In) | (Ft\hat{ }3) | Volume | (Ft) 2007 | (In) 2007 | (Ft\hat{ }3) 2007 |
| IPB1 | 100% | 25.4′ | 2.74″ | 1.344 | 89% | 20.7′ | 2.66″ | 1.344 |
| #427 | 100% | 22.5′ | 2.51″ | 1.007 | 67% | 18.4′ | 2.51″ | 1.007 |
| #470 | 100% | 22.8′ | 2.03″ | 0.719 | 48% | 18.9′ | 2.02″ | 0.719 |
| #490 | 100% | 26.3′ | 2.87″ | 1.507 | 100% | 21.9′ | 2.82″ | 1.507 |
| #494 | 100% | 25.7′ | 2.49″ | 1.113 | 74% | 21.5′ | 2.48″ | 1.113 |

These results show that survival under the conditions experienced at Loxely, Ala. were good for both the controls and transgenics, but as described above the dieback was greater on non transgenic plants.

The same lines were also tested at a number of other sites where temperatures dropped lower and thus a greater freezing stress was encountered. At Washington, La., from July 2006 to November 2007, the temperature data was downloaded from LSU Ag Center which recorded 20 freezing events during which the ambient temperature was equal to or lower than 0° C. (32° F.). The longest freezing event lasted 11.5 hours and the shortest freezing event lasted 0.5 hours. During three of these events the ambient temperature was also recorded to be below or equal to −3.89° C. (25° F.) but above −6.67° C. (20° F.) for a total of 19 hours, the longest lasted 8 hours and the shortest lasted 3 hours. During one of these events the ambient temperature was also recorded to be below or equal to −6.67° C. (20° F.) but above −9.44° C. (15° F.) for a total of two hours. Overall, there were 108 cumulative freezing hours during the freezing events.

Table 12 below summarizes survival, height and growth data of the five transgenic lines and control lines that were assessed in November 2007.

TABLE 12

| | | | | | | Net | | Net Volume |
|---|---|---|---|---|---|---|---|---|
| | | | | Volume | | Height | DBH | Index |
| Line | | Height | DBH | Index | Relative | Growth | Growth | Growth |
| # | Survival | (Ft) | (In) | (Ft\hat{ }3) | Volume | (Ft) 2007 | (In) 2007 | (Ft\hat{ }3) 2007 |
| IPB1 | 36% | 12.0′ | 1.02″ | 0.116 | 100% | 7.7′ | 0.75″ | 0.116 |
| #427 | 100% | 16.2′ | 1.81″ | 0.394 | 96% | 12.6′ | 1.81″ | 0.394 |
| #435 | 100% | 16.5′ | 1.84″ | 0.411 | 100% | 12.5′ | 1.82″ | 0.411 |
| #470 | 90% | 13.2′ | 1.45″ | 0.213 | 52% | 9.3′ | 1.44″ | 0.213 |
| #490 | 100% | 16.5′ | 1.83″ | 0.401 | 97% | 12.6′ | 1.83″ | 0.401 |
| #494 | 100% | 15.4′ | 1.45″ | 0.235 | 57% | 11.8′ | 1.44″ | 0.235 |

At Summerville, S.C., the same lines tested above were planted in July 2006 and assessed in December 2007. From July 2006 to December 2007, the thermometer in the testing field recorded 27 freezing events during which the ambient temperature was equal to or lower than 0° C. (32° F.). The longest freezing event lasted 16.25 hours and the shortest freezing event lasted 0.5 hours. During three of these events the ambient temperature was also recorded to be below or equal to −3.89° C. (25° F.) but above −6.67° C. (20° F.) for a total of 21.5 hours, the longest lasted 11.5 hours and the shortest lasted 2 hours. During one of these events the ambient temperature was also recorded to be below or equal to −6.67° C. (20° F.) but above −9.44° C. (15° F.) for a total of 5.5 hours. Overall, there were 188.75 cumulative freezing hours during the 27 freezing events.

Table 13 below summarizes survival, height and growth data of the five transgenic lines and control lines that were assessed in December 2007.

TABLE 13

| | | | | | | Net | | Net Volume |
|---|---|---|---|---|---|---|---|---|
| | | | | Volume | | Height | DBH | Index |
| Line | | Height | DBH | Index | Relative | Growth | Growth | Growth |
| # | Survival | (Ft) | (In) | (Ft\hat{ }3) | Volume | (Ft) 2007 | (In) 2007 | (Ft\hat{ }3) 2007 |
| IPB1 | 70% | 13.1′ | 0.94″ | 0.100 | 21% | 8.5′ | 0.89″ | 0.100 |
| #427 | 100% | 19.0′ | 1.86″ | 0.479 | 100% | 14.9′ | 1.85″ | 0.479 |
| #435 | 100% | 17.7′ | 1.72″ | 0.430 | 90% | 13.7′ | 1.73″ | 0.430 |
| #470 | 70% | 12.3′ | 1.06″ | 0.226 | 47% | 9.0′ | 1.05″ | 0.226 |
| #490 | 80% | 15.6′ | 1.34″ | 0.253 | 53% | 11.7′ | 1.33″ | 0.253 |
| #494 | 90% | 14.7′ | 1.31″ | 0.296 | 62% | 11.1′ | 1.31″ | 0.296 |

At Burch Fiber Farm, S.C. the same lines tested above were planted in July 2006 and assessed in November 2007. From July 2006 to November 2007, the thermometer in the testing field recorded 47 freezing events during which the ambient temperature was equal to or lower than 0° C. (32° F.). The longest freezing event lasted 15.25 hours and the shortest freezing event lasted 0.25 hours. During 18 of these events the ambient temperature was also recorded to be below or equal to −3.89° C. (25° F.) but above −6.67° C. (20° F.) for a total of 94 hours, the longest lasted 14.25 hours and the shortest lasted 1.5 hours. During 5 of these events the ambient temperature was also recorded to be below or equal to −6.67° C. (20° F.) but above −9.44° C. (15° F.) for a total of 27.25 hours, the longest lasted 11.75 hours and the shortest lasted 2.25 hours. During one of these events the ambient temperature was also recorded to be below or equal to −9.44° C. (15° F.) for a total of 4.25 hours. Overall, there were 327 cumulative freezing hours during the 47 freezing events.

Table 14 below summarizes survival, height and growth data of the five transgenic lines and control lines that were assessed in November 2007.

TABLE 14

| | | | | | | Net | | Net Volume |
|---|---|---|---|---|---|---|---|---|
| | | | | Volume | | Height | DBH | Index |
| Line | | Height | DBH | Index | Relative | Growth | Growth | Growth |
| # | Survival | (Ft) | (In) | (Ft\hat{ }3) | Volume | (Ft) 2007 | (In) 2007 | (Ft\hat{ }3) 2007 |
| IPB1 | 63% | 8.6′ | 0.63″ | 0.047 | 94% | 4.7′ | 0.62″ | 0.047 |
| #427 | 60% | 8.2′ | 0.62″ | 0.036 | 72% | 4.4′ | 0.62″ | 0.036 |
| #435 | 60% | 7.6′ | 0.55″ | 0.050 | 100% | 3.8′ | 0.55″ | 0.060 |
| #470 | 60% | 6.1′ | 0.35″ | 0.011 | 22% | 2.1′ | 0.34″ | 0.011 |
| #490 | 80% | 8.7′ | 0.67″ | 0.041 | 82% | 4.2′ | 0.64″ | 0.041 |
| #494 | 30% | 8.7′ | 0.52″ | 0.042 | 84% | 4.9′ | 0.52″ | 0.042 |

The results obtained from the field test strongly suggest that the Eucalyptus IPB1 line plants carrying the AtCBF2 gene have enhanced survival when exposed to chilling and/or freezing temperature.

### Example 15

### Chilling Stress of Potted Eucalyptus Plants Revealed that Eucalyptus Species have Different Levels of Chilling Tolerance

Chilling stress refers to the low temperature stress between 0° C. to 12° C. Since there is no difference among Eucalyptus species in terms of freezing tolerance, a chilling stress was performed in order to examine the effect of chilling, rather than freezing, on different Eucalyptus species. Potted plants were stressed at 0° C. for 6 days and then returned to the greenhouse. The species tested were E. macarthurii, E. dunnii, E. grandis, and E. camaldulensis. Each pot contained two or three plants. The results of the experiments showed that chilling damage or injury was observed under the stress condition of 0° C. for 6 days. In all cases, the E. camaldulensis plants were heavily damaged, while E. macarthurrii and E. dunnii showed no damage under the same conditions (See Table 15). The chilling tolerance of E. grandis is better than E. camaldulensis, but worse than E. dunnii.

TABLE 15

| Eucalyptus spp | # of tree tested | Observation of leaf damage |
|---|---|---|
| E. camaldulensis | 4 | 90% of young leaves were damaged |
| E. dunnii | 4 | no damage on young leaves |
| E. grandis | 5 | 50% of young leaves were damaged |
| E. macarthurrii | 3 | no damage on young leaves |

### Example 16

### Transgenic Eucalyptus Plants Expressing the CBF2 Gene have Enhanced Resistance to Water Stress

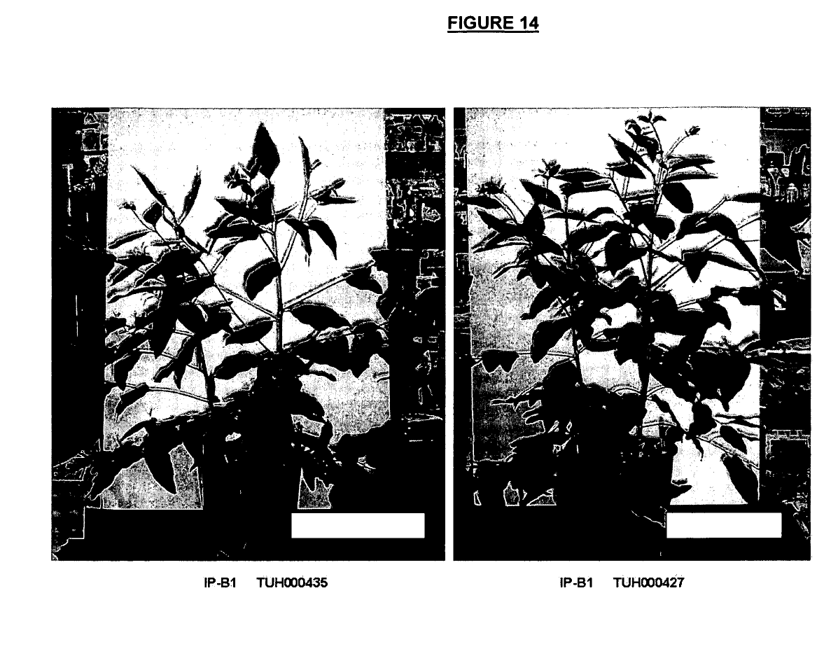

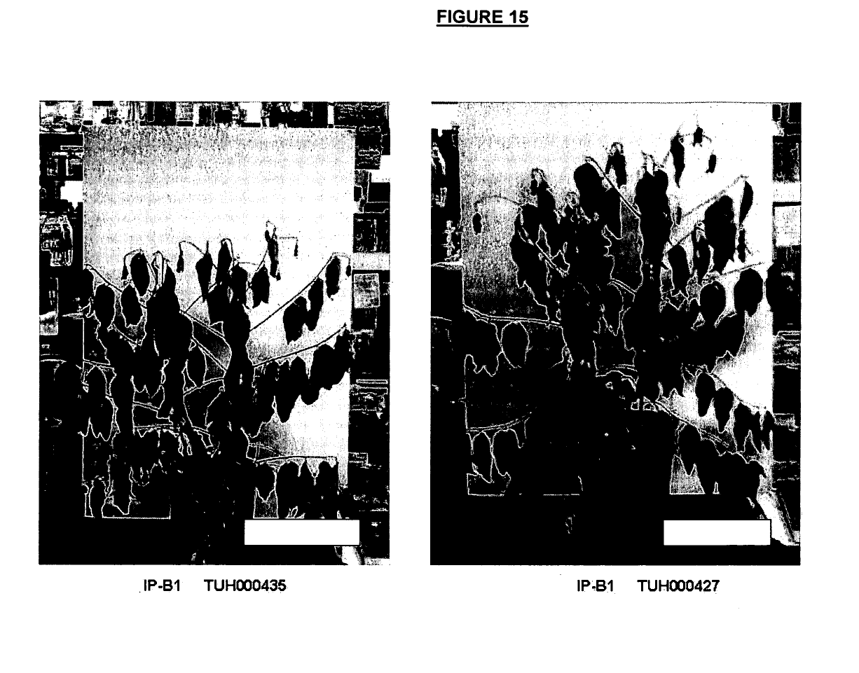

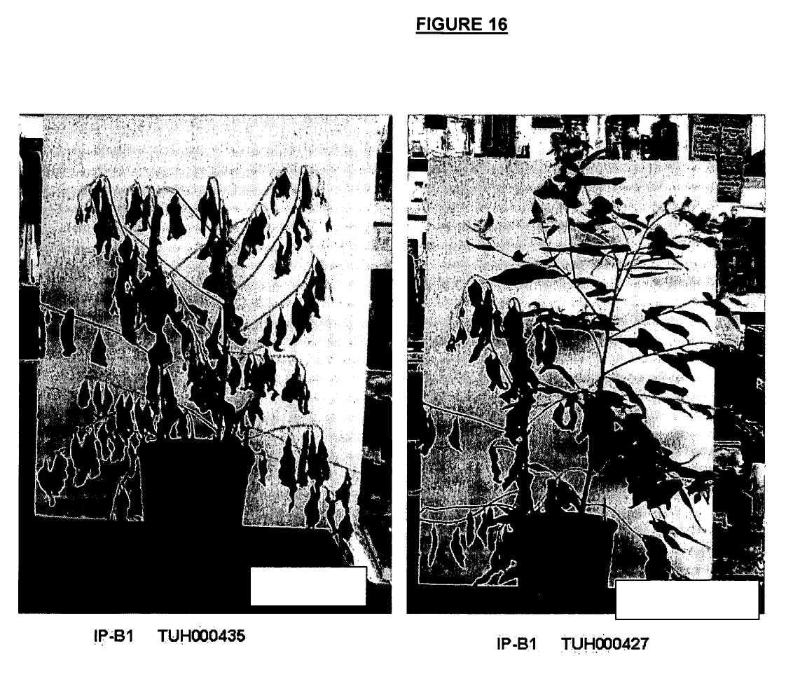

Transgenic IPB1 Eucalyptus plants expressing the CBF2 gene were tested for tolerance to water stress. Two transgenic lines, TUH000427 and TUH000435, were tested. One transgenic IPB1 young plant and one wild-type (WT) IPB1 young plant were grown in a 1-gallon pot (see FIG. 14). At the time of test, the plants in the pots were about 3 ft tall. Temperature and relative humidity were maintained at 70° F. and 65%, respectively. The plants were not watered for eight days. At the end of 8 days, the plants in the pots were severely desiccated as indicated by the wilting of all leaves on the plants (see FIG. 15). No significant differences between the transgenic and WT plants were observed. The soil in the pots was very dry. The plants were then watered. 24 hours after watering there was no significant plant recovery and no differences were detected between the WT and the transgenic plants. The pots were then transferred to a greenhouse and watered regularly for ten days. At the end of the ten day-period, the TUH000427 transgenic line plant had completely recovered from water stress and had grown in size. In contrast, the WT plant in the same pot, as well as the TUH000435 transgenic line plant growing in a different pot, had died (see FIG. 16).

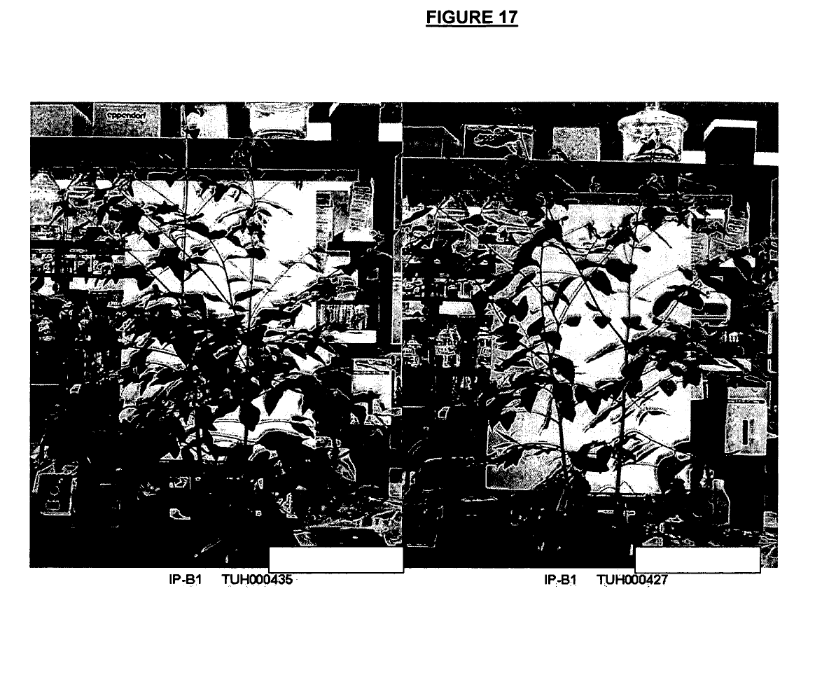

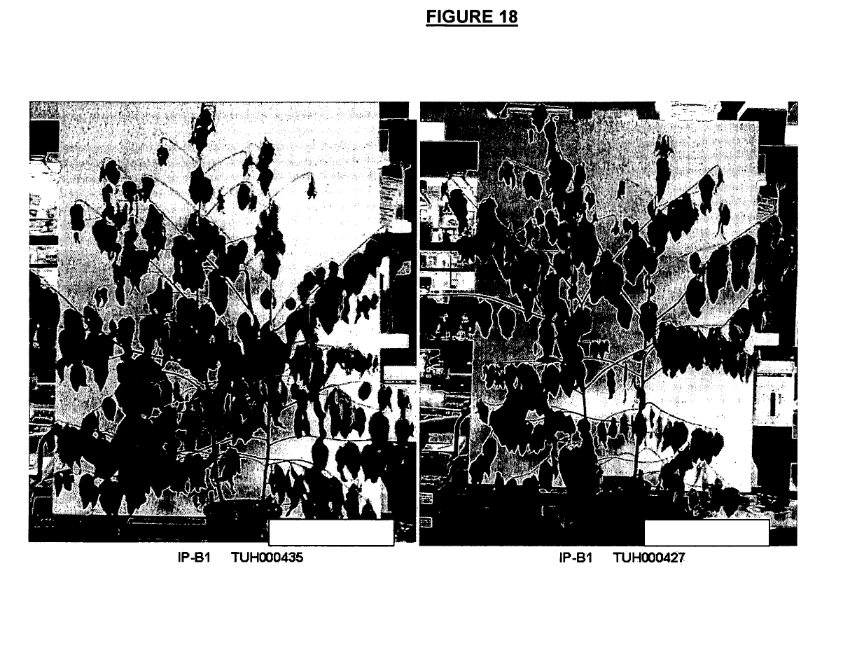

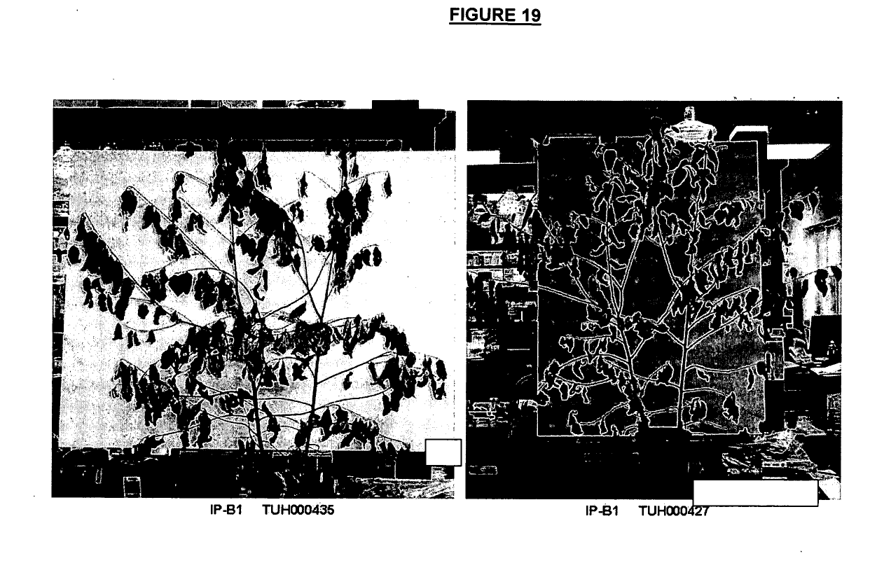

The experiment was repeated with two more pots of the same plants (see FIG. 17). The plants were not watered for eight days. At the end of the 8-day period, the plants were wilting (see FIG. 18). The plants were then watered and 24 hours later were transferred to a greenhouse. Both the TUH000427 transgenic plant and the WT plant growing in the same pot survived the stress and recovered. The TUH000427 transgenic plant recovered faster and suffered less leaf damage than the WT plant in the same pot (see FIG. 19). Both the TUH000435 transgenic plant and the WT plant growing in the same pot died after 10 days, although the leaves of the transgenic plant remained green for a longer period of time and had delayed signs of wilting when compared to the WT plant. (see FIG. 19).

These results indicate that IPB1 Eucalyptus plants expressing the CBF2 gene have enhanced tolerance to water stress.

1.-159. (canceled)

160. A DNA construct comprising at least one isolated polynucleotide comprising a sequence selected from the group consisting of SEQ ID NO: 1, SEQ ID NO: 3, SEQ ID NO: 5, and SEQ ID NO: 7, or a fragment or variant thereof which is at least 90% identical to the nucleotide sequence of SEQ ID NO: 1, SEQ ID NO: 3, SEQ ID NO: 5, or SEQ ID NO: 7 operably linked to a promoter, wherein the isolated polynucleotide, fragment or variant encodes a CBF polypeptide and wherein the promoter drives the expression of the CBF polypeptide in a tree upon exposure of the tree to a stress condition for a period of time, while reducing undesirable effects associated with the expression of the CBF polypeptide in the tree.

161. The DNA construct of claim 160, wherein the stress condition is selected from the group consisting of a freezing temperature, water stress and high-salt conditions.

162. The DNA construct of claim 161, wherein the stress condition is a freezing temperature from about 0° C. to about −30° C. and the period of time is from about 2 hours to about 72 hours.

163. The DNA construct of claim 161, wherein the stress condition is water stress and the period of time is from about one day to about 10 days or up to the wilting point.

164. The DNA construct of claim 160, wherein the promoter is Arabidopsis thaliana rd29A promoter or a dehydrin promoter comprising a nucleic acid sequence selected from the group consisting of SEQ ID NO: 9, SEQ ID NO: 10 and SEQ ID NO: 11.

165. An isolated tree cell comprising the DNA construct of claim 164.

166. A transgenic tree comprising the isolated tree cell of claim 165, wherein the transgenic tree is selected from the group consisting of eucalyptus, poplar, citrus, papaya, avocado, nutmeg, pistachio, kiwi and jojoba.

167. The transgenic tree of claim 166, wherein the eucalyptus is an eucalyptus species selected from the group consisting of Eucalyptus amplifolia, Eucalyptus benjensis, Eucalyptus benthamii, Eucalyptus calmaldulensis, Eucalyptus dorrigoensis, Eucalyptus dunnii, Eucalyptus globulus, Eucalyptus grandis, Eucalyptus gunnii, Eucalyptus macarthurii, Eucalyptus nitens, Eucalyptus urophylla, Eucalyptus viminali, Eucalyptus grandis×Eucalyptus urophylla, and hybrids thereof.

168. A method for producing the transgenic tree of claim 166 comprising (a) transforming a tree cell with a DNA construct that comprises at least one isolated polynucleotide comprising a sequence selected from the group consisting of SEQ ID NO: 1, SEQ ID NO: 3, SEQ ID NO: 5 and SEQ ID NO: 7, or a fragment or variant thereof which is at least 90% identical to the nucleotide sequence of SEQ ID NO: 1, SEQ ID NO: 3, SEQ ID NO: 5, or SEQ ID NO: 7, operably linked to a promoter, wherein the isolated polynucleotide, fragment or variant encodes a CBF polypeptide and wherein the promoter drives the expression of the CBF polypeptide in a tree upon exposure of the tree to a stress condition for a period of time, while reducing undesirable effects associated with the expression of the CBF polypeptide in the tree, to produce a transformed tree cell; (b) culturing the transformed tree cell under conditions that promote growth of a tree, wherein the CBF polypeptide is expressed in the transformed tree cell, and wherein the tree is a transgenic tree that exhibits stress tolerance upon exposure to a stress condition for a period of time.

169. The method of claim 168, further comprising the step of cold-acclimating the transgenic tree prior to exposure to the stress condition.

170. The method of claim 168, wherein the stress tolerance is freezing, water stress or high-salt tolerance and the stress condition is selected from the group consisting of a freezing temperature, water stress and high-salt conditions.

171. The method of claim 170, wherein the freezing temperature is from about 0° C. to about −30° C. and the period of time is from about 2 hours to about 72 hours.

172. The method of claim 170, wherein the stress condition is water stress and the period of time is from about one day to about 10 days or up to the wilting point.

173. A method of making wood pulp from the transgenic tree of claim 166 comprising (a) removing the bark from the wood of the transgenic tree; (b) separating the cellulose fibers in the wood; (c) dissolving the lignin in the wood to obtain wood pulp; and (d) bleaching the wood pulp to produce paper.

174. A method of making veneer from the transgenic tree of claim 166 comprising (a) cutting the transgenic tree into logs; (b) removing the bark from the logs of the transgenic tree; (c) exposing the logs to high humidity for 48 hours; and (d) slicing the veneer from the logs to obtain sliced veneer.

175. A method of making tall oil from the transgenic tree of claim 166 comprising (a) digesting the wood of the transgenic tree under pressure with sodium hydroxide and sodium sulfide; (b) condensing the volatilized gases to yield sulfate turpentine; (c) concentrating the pulping solution thus obtained; (d) allow the insoluble soaps to be skimmed from the surface; and (e) acidifying the skimmed soap to produce crude tall oil.

176. A method of producing biofuel from the transgenic tree of claim 166 comprising the step of converting the biomass of the transgenic tree into fuel.

177. A method of producing bioenergy from the transgenic tree of claim 166 comprising the step of producing bioenergy from the transgenic tree.

178. A method of enhancing freezing tolerance in a tree comprising (a) transforming a tree cell with a DNA construct that comprises at least one isolated polynucleotide comprising a sequence selected from the group consisting of SEQ ID NO: 1, SEQ ID NO: 3, SEQ ID NO: 5 and SEQ ID NO: 7, or a fragment or variant thereof which is at least 90% identical to the nucleotide sequence of SEQ ID NO: 1, SEQ ID NO: 3, SEQ ID NO: 5, or SEQ ID NO: 7, operably linked to a promoter, wherein the isolated polynucleotide, fragment or variant encodes a CBF polypeptide and wherein the promoter drives the expression of the CBF polypeptide in a tree upon exposure of the tree to a stress condition for a period of time, while reducing undesirable effects associated with the expression of the CBF polypeptide in the tree, to produce a transformed tree cell; (b) culturing the transformed tree cell under conditions that promote growth of a tree to produce a transgenic tree; (c) subjecting the transgenic tree to cold acclimation; (d) exposing the transgenic tree to a freezing temperature from about 0° C. to about −30° C. for a period of time from about 2 hours to about 72 hours; and (e) allowing the transgenic tree to recover under conditions that promote growth of the transgenic tree.

179. The method of claim 178, wherein the promoter is Arabidopsis thaliana rd29A promoter.

180. The method of claim 178, wherein the transgenic tree is selected from the group consisting of eucalyptus, poplar, citrus, papaya, avocado, nutmeg, pistachio, kiwi and jojoba, and expresses a CBF polypeptide comprising an amino acid sequence selected from the group consisting of SEQ ID NO: 2, SEQ ID NO: 4, SEQ ID NO: 6, and SEQ ID NO: 8, or a fragment or variant thereof which is at least 90% identical to the nucleotide sequence of SEQ ID NO: 2, SEQ ID NO: 4, SEQ ID NO: 6, or SEQ ID NO: 8.


SEQUENCE LISTING (19 sequences)

SEQ ID NO: 1 - DNA, 826 bp, Artificial Sequence
  Description of Artificial Sequence Synthetic nucleotide construct
atggacttctccgacaaggaagtgcagctcgcgtccgaccacccgaagaagcccgccgggaggaagaagttccgggagacccgccaccccgtgtaccgcggggtgcgtctgcgcgactcgggcaagtgggtctgcgaggttcgcgagcccaaaaagaagtcgaggatctggctcggcaccttccctactgtggagatggcagcgagggcgcatgacgtggcagcgctcgagctgagaggccagtctgcctgcctcaacttcgcagactctgcgtggcggctgcccaagccggcatcggcggatcccaaggacatacagaaggcggcggccaaggctgcagaggcattccagccgctggaatccgaggacataatgtcaggggacgagaagaagctgcattcggaagagggagtgtcgtacgacgaggaggacatcttggggatgccagggttgatcgccgatatggcggaggggatgctcttgtctcccccacaatgcggcgaagatatatatggcggagaggacgacgggaacttggacgcatacgcgtcattatggagccattccatctgaccatttctcaatttcacttgtacatagaagctgtttaatagagtgcggaagccactttttgtgatgtgccattagataaagagcatatcgatgaagcacgggtcttccgatgttgggccgccgatagagcggatgggaaagtgaaacaaagcgcaaaagtaggctgcgttcacagcaacatcgggcgttgtttatagttaataaagaaaccgcccgagccatcatgaatttgagagaagtggttttcaaactt

SEQ ID NO: 2 - protein, 190 aa, Artificial Sequence
  Description of Artificial Sequence Synthetic polypeptide
MDFSDKEVQLASDHPKKPAGRKKFRETRHPVYRGVRLRDSGKWVCEVREPKKKSRIWLGTFPTVEMAARAHDVAALELRGQSACLNFADSAWRLPKPASADPKDIQKAAAKAAEAFQPLESEDIMSGDEKKLHSEEGVSYDEEDILGMPGLIADMAEGMLLSPPQCGEDIYGGEDDGNLDAYASLWSHSI

SEQ ID NO: 3 - DNA, 989 bp, Artificial Sequence
  Description of Artificial Sequence Synthetic nucleotide construct
atggactcttcctcttatatctcccaccccaactcctttagcttcgacttcgccgagccgcctttctcgttgctgctgtcccacgaccggagcgcagcgcccgggaacttctctggggaggaggtgcgcctcgcgtccgaccacccgaagaagcccgccggcaggaagaagttccgggagacacgccaccccgtgtaccgtggggtgcggctgcgcgactcgggaaagtgggtctgcgaggtccgcgaacccagaaagaagtcgaggatctggcttggcaccttccccaccgcagacatggcagcgagggcgcacgatgtggcggcgctcgcactaaggggccagtccgcctgcctcaacttcgcagactccgcgtggcggctgccggtgccggcgtcacccaacaccaaagacatacagaaggcagcaaccaaggccgcggaggcattccagctgctggaatccgaggatgtgatgtcggggaacgagaagaagttgcattcggaagagggagtgttgtacgacgaggaggacatcttcgggatgccggggttgctcgccaatatggcggaggggatgctcttgtctccaccggaatgcagcggagatttatatgccggactggacgacgggaacttggacgcatacgcgtcattatggagctattccatgtgaccatttctcaatttcactttaagtgttttgtcattagggacgactttgatgccccacttagtaagtgttttaatagagtgcggataaaagttgaagcgtatgaacacagatattttcggaagccatttttgtggagtgccattagataaaaaggctgtgttcaccgcaacatcgggcgtttgcatatagttaataaagaaactgcccgagtcaacatgaatttaagagaagtggttttcagacttacccaatttatcttcgaatgtatcatgtctatggcaaaggtgcgccaacgaaattcgtatgggttcttt

SEQ ID NO: 4 - protein, 224 aa, Artificial Sequence
  Description of Artificial Sequence Synthetic polypeptide
MDSSSYISHPNSFSFDFAEPPFSLLLSHDRSAAPGNFSGEEVRLASDHPKKPAGRKKFRETRHPVYRGVRLRDSGKWVCEVREPRKKSRIWLGTFPTADMAARAHDVAALALRGQSACLNFADSAWRLPVPASPNTKDIQKAATKAAEAFQLLESEDVMSGNEKKLHSEEGVLYDEEDIFGMPGLLANMAEGMLLSPPECSGDLYAGLDDGNLDAYASLWSYSM

SEQ ID NO: 5 - DNA, 690 bp, Artificial Sequence
  Description of Artificial Sequence Synthetic polynucleotide
atggaccacttcttctcttcttactccgatcccagctcctgcagcctcgactttgctgaagcgtcgtcctcctcgtcgccgctgtccgatggcaggagtgctatggtgcccgggaacttttctgatgaggaggtgctcctggcgtcgcaccagccgaagaagcgcgccgggcggaagaagttccaggagacgcgccaccccgtgtaccgcggggtgcggcggcgaagctcgggcaagtgggtctgcgaggtccgcgagcccaacaagaagtcgaggatctggctgggcaccttccccaccgcggagatggctgcgagggcgcacgacgtggcggcgctcgcgctgaggggccggtccgcctgcctcaacttcgcggactccgcgtggcggctgcccgcgccggcgtcggcggacgcaaaggacatacagcaggcggcggcccaggctgcggaggcgttccggccggcggagtccgaggctgaggacgtgatgtcggggtacgagaagaagtcgccttcggaggagggaatgctgtacgacgacgaggacgtcttcgggatgccggggttactcacgaatatggcggaggggatgctcttgcctccaccacaatgcggcggagatgggtacggcggagaggacgacgggaatctggatgcatacgtgtcgttatggaactattccatgtag

SEQ ID NO: 6 - protein, 229 aa, Artificial Sequence
  Description of Artificial Sequence Synthetic polypeptide
MDHFFSSYSDPSSCSLDFAEASSSSSPLSDGRSAMVPGNFSDEEVLLASHQPKKRAGRKKFQETRHPVYRGVRRRSSGKWVCEVREPNKKSRIWLGTFPTAEMAARAHDVAALALRGRSACLNFADSAWRLPAPASADAKDIQQAAAQAAEAFRPAESEAEDVMSGYEKKSPSEEGMLYDDEDVFGMPGLLTNMAEGMLLPPPQCGGDGYGGEDDGNLDAYVSLWNYSM

SEQ ID NO: 7 - DNA, 989 bp, Artificial Sequence
  Description of Artificial Sequence Synthetic nucleotide construct
atggcagcccccgggaacttctccgacgaggaggcgcgcctcgcgtcccaccacccaaagaagcgcgccgggaggaagaagttccgggagacgcgccaccccgtgtaccggggagtgcggctgcgtgactcgggcaagtgggtctgcgaggtccgcgagcccagaaagaagtcgaggatctggctcggcaccttccctactgtggagatggcggcgagggcgcacgatgtggcagcgctcgcgctgaggggccggtctgcctgcctcaacttcgcagactccgcgtggcggctgcccgtgccagcgtcggcagacaccaaggacatacagaaggcggcggccaaggccgcggaggcattccagccagtggagtccgagtccgaggacgtgatgtcgggggacgagaagaagtcgccttcggaggagggaatgctgtacgacgacgaggacgtcttcgggatgccggggttgctcatgaatatggcggaggggatgctcttgcctccaccacgatgcggcggagatgggtacggcggagaggacgacgggaatctggatgcatacgtgtcgttatggagctattccatgtagtcatttctcaagttcagttatactttttgtggttagggacgactgggatgccgtactaaaatattttttgatggagtgcggatgaaaggcgaaggtgggtacgaaagcagaaggtattttcggaagccaatttttggggcatgccgttaggtagataatagagactgcacggctcttctgatgttgggcctctgatggaagaataaaagtaggccatgttcacggcagcatcgggcgttgcatatagttgataaagagactgcccgagtcatcatgatcttcgaatgtaaatcatgtaccatgtacaaggcaaagagtgtgctcttgtaattcttatggtttgtttgggttcaaatgctttttgctctggcccattaagacgataaattgaccatccc

SEQ ID NO: 8 - protein, 196 aa, Artificial Sequence
  Description of Artificial Sequence Synthetic polypeptide
MAAPGNFSDEEARLASHHPKKRAGRKKFRETRHPVYRGVRLRDSGKWVCEVREPRKKSRIWLGTFPTVEMAARAHDVAALALRGRSACLNFADSAWRLPVPASADTKDIQKAAAKAAEAFQPVESESEDVMSGDEKKSPSEEGMLYDDEDVFGMPGLLMNMAEGMLLPPPRCGGDGYGGEDDGNLDAYVSLWSYSM

SEQ ID NO: 9 - DNA, 620 bp, Eucalyptus dunnii
agcttctaatacgactcactataggggtccgatcaaggacatccatccaacgacgcaattccacgtttccccttcctagtggctaggacagcgccacccacctcccctcgcctccctctctctctactactttctctctccttctttttcccaccgtttgaccgtgcctttccagctcccctcgctcgcgtacaccgtaagctcacgcgccatccgacgaaagcggggccgctcgtcgagagcagcgcgggcgcgtgcgttccctcggcgcgtcggcccgtggcgataccctttttgggtcggctgtccgacttcctctcttaacgcaccacgtcggttaagaagcgggagcggattggccgccccctctaggtgggcccacttcagatctctcccagaattccttccttcttcatgcttaatcgcgtcatttccgtgtcaattctatgctataaaaaccgcccaaactcactcctcagcatcatcacacagaaaacccatcgagaaacaccagaaaaagctccgaggaatacgtctgtcgtcttttatcattcgcattaccgcatacttcgaagatcgccggtcgttgatcgcagcagcggtgtgtgttgagagccatg

SEQ ID NO: 10 - DNA, 620 bp, Eucalyptus macarthurrii
agcttctaatacgactcactatagggcttagatcaaggacatccatccaacgacgcaattccacgtttccccttcctagtggctaggacagcgccacccacctcccctcgcctccctctctctctactactttctctctccttctttttcccaccgtttgactgtgcctttccagctcccctcgctcgcgtacaccgtaagctcacgcgccatccgacgaaagcggggccgctcgtcgagagcagcgcgggcgcgtgcgttccctcggcgcgtcggcccgtggcgataccctttttgggtcggctgtccgacttcctctcttaacgcaccacgtcggttaagaagcgggagcggattggccgccccctctaggtgggcccacttcagatctctcccagaattccttccttcttcatgcttaatcgcgtcgtttccgcgtcaaatctatgctataaaaaccgcccaaactcactcctcagcatcatcacacagaaaacccatcgagaaacaccagaaaaagctccgaggaatacgtctgtcgtcttttatcattcgcattaccgcatacttcgaagatcgccggtcgttgatcgcagcagcggtgtgtgttgagagccatg

SEQ ID NO: 11 - DNA, 918 bp, Artificial Sequence
  Description of Artificial Sequence Synthetic nucleotide construct
gtggagccatcatgtcaattccaggttttaattaatgctactaagggtcatgctataaagccgttaacaaatcgttgtgatgatagaaagaggctgctgtaatattaatactgctcagtttcaaggagacgtgtgcctttacgtgtctatagcgttcatctccgtccttctagaagaatactttttttttccttcgagggtaaggaaaaggcaaataaaaattacatatttaatcttaattagcaatagttactttcctattaaatacagacacggggattccaagcatttgcccgacggccaaaaaaaagatattatccaccattctatcttaattattacctctaattattatgcttatcctcctacaagataattaacgaattatatttcttttatgaatacacatttaacttgaattacttgaatagattgtatatgattatccgatcaagaggaaggtactgttaatattaatattttatctgatttatttattataatttgtcaaattattaatcattatactgatttatcaagttatatttaaaaaataataaattttgtttagcataaattttaaaaaataattttgatatatatattttatagagcttatagataactattgcttttggggaaaaaaacgcgtaggatattattcatagagaatctaaaaaaagggaaacctcttgaacccctcatttaaagtctccatgtaaaagacggctcgcgcggctcaggaattttcctgtcactttccttcgaaccaaacagaattgtatatatacccaacgtttctcaattgactcttatagaaaacccccgaaaaagaacaatctttaaggaaaaaattctctcttattcttgataacaataccttgatcgtttttgtttgtttgtagaggaaaatagtac

SEQ ID NO: 12 - DNA, 11078 bp, Artificial Sequence
  Description of Artificial Sequence Synthetic nucleotide construct
gtttacccgccaatatatcctgtcaaacactgatagtttaaacttttaattaaggtaccgggccccccctcgaggtcgacggtatcgataagcttggttgctatggtagggactatggggttttcggattccggtggaagtgagttgggaggcagtggcggaggtaagggagttcaagattctgggaactgaagatttggggttttgcttttgaatgtttgtgtttttgtatgatgcctctgtttgtgaactttgatgtattttatctttgtgtgaaaaagagattgggttaataaaatatttgcttttttggataagaaactcttttagcggcccattaataaaggttacaaatgcaaaatcacgttagcgtcagatatttaattattcgaagatgattgtgatagatttaaaattatcctagtcaaaaagaaagagtaggttgagccagaaacagtgacatctgttgtttgtaccatacaaattagtttagattattggttaacatgttaaatggctatgcatgtgacatttagaccttatcggaattaatttgtagaattattaattaagatgttgattagttcaaacaaaaattttatattaaaaaatgtaaacgaatattttgtatgttcagtgaaagtaaaacaaattaaattaacaagaaacttatagaagaaaatttttactatttaagagaaagaaaaaaatctatcatttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcgggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagtcacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttggcgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaccatggactcattttctgccttttctgaaatgtttggctccgattacgagtctccggtttcctcaggcggtgattacagtccgaagcttgccacgagctgccccaagaaaccagcgggaaggaagaagtttcgtgagactcgtcacccaatttacagaggagttcgtcaaagaaactccggtaagtgggtgtgtgagttgagagagccaaacaagaaaacgaggatttggctcgggactttccaaaccgctgagatggcagctcgtgctcacgacgtcgccgccatagctctccgtggcagatctgcctgtctcaatttcgctgactcggcttggcggctacgaatcccggaatcaacctgtgccaaggaaatccaaaaggcggcggctgaagccgcgttgaattttcaagatgagatgtgtcatatgacgacggatgctcatggtcttgacatggaggagaccttggtggaggctatttatacgccggaacagagccaagatgcgttttatatggatgaagaggcgatgttggggatgtctagtttgttggataacatggccgaagggatgcttttaccgtcgccgtcggttcaatggaactataattttgatgtcgagggagatgatgacgtgtccttatggagctattaaaattcgatttttatttccatttttggtattatagctttttatacatttgatccttttttagaatggatcttcttctttttttggttgtgagaaacgaatgtaaatggtgcggccgctctagacaggcctcgtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactggtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcaggagcccgggctctcgagtaaaacataattttggcagtaaaaaagtgaattctattgttttgaaaacaaaacaaaatacaggaagcgtgattgtggggttgttgttgaacttgcccgggcaaaagaagaatgattagcggtagaggagttagtagttacgttcaactaaatgcgtgactaaattatttatcctccgccatggaagcaggtgattcacacacaacttgctgcacacattgctctcaaacctttcctataaatatccgtagcaggggctgcgatgatacacaacgcatttaatcaaactactttgattactttctgtgggttctactttctttgaatagtcagttctgctgtttttagaagatttatggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggacgagtatcagacctttacaaaaatcagataacgaaaaaaacggcttccctgcgggaggccgtttttttcagctttacataaagtgtgtaataaatttttcttcaaactctgatcggtcaagagctcttctgagagacaatacatacatgtctctgatgttgtaactttactaccaaaacctataaagattggcttatttcgttctattggatatgtatcatcattactggtaaatcaagtttctttctaataatgtagaagatcagaaaatccataagaagatatcaacatttgagttctatggtaaattgaattatatcaacttagttgcaatgattcattcttgactgatgcattgatggcttatcaaaccagtttcaaaattcgattagatagggcccatttaaatgcggccgcttggcgcgccgtcaacggatcaggatatccttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcacgtaggggggatccactagttctagagcggccgtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagttgtctatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattgtggtttgtgtttccatattgttcctctcccattgatcgtattaagaaagtatgatggtgatgtcgcagccttccgctttcgcttcacggaaaacctgaagcacactctcggcgccattttcagtcagctgcttgctttgttcaaactgcctccattccaaaacgagcgggtactccacccatccggtcagacaatcccataaagcgtccaggttttcaccgtagtattccggaagggcaagctcctttttcaatgtctggtggaggtcgctgatacttctgatttgttccccgttaatgactgcttttttcatgtgcggctcctttcttatgattttattctatcagtttaccatttttctcttcagaaatggccggattctgccccggtttcagctggacgatgtcctcctgtctcggacggctgcaaattggaccacaattatggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggatctggggaaccctgtggttggcatgcacatacaaatggacgaacggataaaccttttcacgcccttttaaatatccgattattctaataaacgctcttttctcttag

SEQ ID NO: 13 - DNA, 14947 bp, Artificial Sequence
  Description of Artificial Sequence Synthetic nucleotide construct
cgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccataattgtggtccaatttgcagccgtccgagacaggaggacatcgtccagctgaaaccggggcagaatccggccatttctgaagagaaaaatggtaaactgatagaataaaatcataagaaaggagccgcacatgaaaaaagcagtcattaacggggaacaaatcagaagtatcagcgacctccaccagacattgaaaaaggagcttgcccttccggaatactacggtgaaaacctggacgctttatgggattgtctgaccggatgggtggagtacccgctcgttttggaatggaggcagtttgaacaaagcaagcagctgactgaaaatggcgccgagagtgtgcttcaggttttccgtgaagcgaaagcggaaggctgcgacatcaccatcatactttcttaatacgatcaatgggagaggaacaatatggaaacacaaaccacaattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgatagacaactgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccacggccgctctagaactagtggatcccccctacgtgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatctgttaatcagaaaaactcagattaatcgacaaattcgatcgcacaaactagaaactaacaccagatctagatagaaatcacaaatcgaagagtaattattcgacaaaactcaaattatttgaacaaatcggatgatatttatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaaggatatcctgatccgttgacggcgcgccaagcggccgcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcacggataacagagtctttatattaaacgaaatggtattgcagtgccggccaggacaccagtattggcttcaactgaccgacgaaaagaaaaatataacataagagggtctgaaacagacaaacaacactaacacatacataaacacaatttattcgtgagaaatacgacaaacactgaacatcacccgaatttattatccacaaataacacccatcggatcgatatttgatttcacatgctattgtaatgtatttattgtttcaattccgaattagacaaagtgcttaaagctctcttttcggattttttttttcattaatgtataataattgcggacattacaatatactgtacaacgtgatttgagcttgatgaattacaagattggaagaacttcgaagacaaaaaaaaaatcgatcgatactagtgggcccctcgagatcgatactagtgggcccctcgagatcgataagcttccagatttcgatttcgaacccggaaagtgtgcccactacaaagtgtgaacccaatttaatcagaatattactaaatctagttttcttcaaagatgtcaaagcccaatcttttatcagagttgtataaatttaagatcatataattattcttatttttaatttaaaattggtacccgtgattaactttctacacaactcgtatgagtgactcgatataaacttattagaaaattatatatatacctataatatttaaatcctagagaatagagttaaatttatttatttacaaatttgttaagaatgttctctacgtgtttagtcttaaaatatgttttcaaacacaactcataaaattaaatggagtatcttccaaatcataaggtctccaagtcgaatttgtaaaaaactataaaaaaataaaaaataataataataaaaaaaacatctactatgacatatgtactataattaaataaggaggccgcaataattctttattggttactaaaacttaagatatttcttttggctaaaaaaagcttatagtactttataaaactgtttgaacatataaaagaaataaaaacttacattcttggcagcagtgctgatcaggggtaccgaattactgctggacgaattcctgcagatcgattttttttttgtcttcgaagttcttccaatcttgtaattcatcaagctcaaatcacgttgtacagtatattgtaatgtccgcaattattatacattaatgaaaaaaaaaatccgaaaagagagctttaagcactttgtctaattcggaattgaaacaataaatacattacaatagcatgtgaaatcaaatatcgatctgcagcccaattcctgcagcccgggggatccggcgatcgaaatactgaaaataaaaacctttcaattgattcaatcccaatccgaacctgatcaaattctccgcagaaacaggcgaaaaatggcggaaaacgtgtctgacaaatgtagcctgcagtgggaagaattgaatgagctgggggctactgagaaggaagcagaggccggctttttatattggattttcctgtgcagtacattgtatagaaagccggcctggaattgttggtgaagagtcaagtggcgtatggttgaataatgggcaaccacggcaggcgatcgagtgagtgggtggagggtcccgtggccaccaactctgcgatgtgaaggagcggttggatttttgacggagattatgactttttccattgacgtctacgttactacctaccccgaaaactccattcggacaactcgccggtaatacgccgtctacctacccccatcccggctaggccaacacccgcgctctctcgcccaccgcttgtttgaccgatcacccagacatttcattcgctgttttcggaccgcatttgtcagtcagttcacaccagggtggaaattagaattggttagattacaataagaaaaatgccttagccaatagcatgggatacaaatctgaaatcccattaaattggtgtttaaaatgtgtttttattatataatggatttagtatcttcaaaaaccgaagatacttggattactgaaagtttataactcaaaccttcattccaataaatcccagagcaaatatgtctggaaaatctctaaaatgtataaaaaaatatattattgggtaaaaaaattctcaatttccaaaaatatggagttcgaactatattttgggagattgagagaaatatttgggattttttttgtctaatttccaatccacaaatgacatgatgagctttaatgtaatgattttgaaatttgtttggaaattaatagtccctagacgagaacattctatatttctcttatacaatataatttccaatccacaaatgacatgatgagctttaatgtaatgattttgaaacttgtttggagattaatagtccctggacgagaacattctatatttcttggatacgatataaagatattttctatgtaaaaaaattaaaaaataagaattaattaatgagatcttaattacgggccatgaggttttttattttattggaaactggttattgtctttcttgaaatcataatgttgataatgttttgtaatttaaattttatttgagatttttggtttttatacaaattggttaatctattccatgaagttttattatttaaattgtattttgaatgcaaaatagatttaatattttggaaaatatttggaatatttgacttcaaaagtttattaatttaaaattaattttttctaaattttaactaaatataagataaaatttaccaacaaattttttaaaaataatagtgtttaattattttagattttgaaatttttactctaaatttaacaaaacaattttttatccaattataatttttgttattcaaaataaaattttaattacatatataaatttaacatcttaaaaaatccataaaacactcattcacaactcaatttatagttcttaccaaccttttccaaattttaagtaaactcaacccaaaaatatatatatatatatatatttttaaaaactccataacatatcacaaataaattactaaaaaaatcccaaaatcacttattaaaacacccttattccataaaataaaagatacgttaattggattcgaactataaaactaatgagttaatgagacgacaaactcattcttaaaaggtagagtttcaagtttgtttatgttttcaaaatttttgtgatcagcgtccttttaaatgtactcacattttcagtgtttagctttattttaaagttttgatgaattttgtataaaaagtttgtttattttgaagttttaataaattttgtataatatttttttttaaatagttaagtttatttatttattaatctattttaattttgttggaggaggttgtgattgaagaagttgtgattaaagtgtattgttgaaagaaattctttatattaagggtttaagtttgaattttgtgaacacaattataattatgttaaagatgaaaattttgttttcttttatttttgtaaatccttcttgttttgagatgaatttatgaataaatgtcaccacccggccgcggccgcatttaaatgggccctatctaatcgaattttgtaaactggtttgataagccatcaatgcatcagtcaagaatgaatcattgcaactaagttgatataattcaatttaccatagaactcaaatgttgatatcttcttatggattttctgatcttctacattattagaaagaaacttgatttaccagtaatgatgatacatatccaatagaacgaaataagccaatctttataggttttggtagtaaagttacaacatcagagacatgtatgtattgtctctcagaagagctcttgaccgatcagagtttgaagaaaaatttattacacactttatgtaaagctgaaaaaaacggcctcccgcagggaagccgtttttttcgttatctgatttttgtaaaggtctgatactcgtccgttgttttgtaaatcagccagtcgcttgagtaaagaatccggtctgaatttctgaagcctgatgtatagttaatatccgcttcacgccatgttcgtccgcttttgcccgggagtttgccttccctgtttgagaagatgtctccgccgatgcttttccccggagcgacgtctgcaaggttcccttttgatgccacccagccgagggcttgtgcttctgattttgtaatgtaattatcaggtagcttatgatatgtctgaagataatccgcaaccccgtcaaacgtgttgataacctgtgccataaatcttctaaaaacagcagaactgactattcaaagaaagtagaacccacagaaagtaatcaaagtagtttgattaaatgcgttgtgtatcatcgcagcccctgctacggatatttataggaaaggtttgagagcaatgtgtgcagcaagttgtgtgtgaatcacctgcttccatggcggaggataaataatttagtcacgcatttagttgaacgtaactactaactcctctaccgctaatcattcttcttttgcccgggcaagttcaacaacaaccccacaatcacgcttcctgtattttgttttgttttcaaaacaatagaattcactttttactgccaaaattatgttttactcgagagcccgggctcctgcaggcgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaagctctagctagaggatccggtacgaggcctgtctagagcgcaccatttacattcgtttctcacaaccaaaaaaagaagaagatccattctaaaaaaggatcaaatgtataaaaagctataataccaaaaatggaaataaaaatcgaattttaatagctccataaggacacgtcatcatctccctcgacatcaaaattatagttccattgaaccgacggcgacggtaaaagcatcccttcggccatgttatccaacaaactagacatccccaacatcgcctcttcatccatataaaacgcatcttggctctgttccggcgtataaatagcctccaccaaggtctcctccatgtcaagaccatgagcatccgtcgtcatatgacacatctcatcttgaaaattcaacgcggcttcagccgccgccttttggatttccttggcacaggttgattccgggattcgtagccgccaagccgagtcagcgaaattgagacaggcagatctgccacggagagctatggcggcgacgtcgtgagcacgagctgccatctcagcggtttggaaagtcccgagccaaatcctcgttttcttgtttggctctctcaactcacacacccacttaccggagtttctttgacgaactcctctgtaaattgggtgacgagtctcacgaaacttcttccttcccgctggtttcttggggcagctcgtggcaagcttcggactgtaatcaccgcctgaggaaaccggagactcgtaatcggagccaaacatttcagaaaaggcagaaaatgagtccatggtccaaagatttttttctttccaatagaagtaatcaaaccctttattcctgatgattgttttctagtttgcatatttgtgagtaaaacagaggagggtctcactaagtttatagagagactgagagagataaagggacacgtatgaagcgtctgttttcgtggtgtgacgccaaagtcattttgctctctacgcgtgtctgtgtcggcttgatctttttttttgctttttggaactcatgtcggtagtatatcttttatttattttttctttttttcccttttctttcaaactgatgtcggtatgatatttattccatcctaaaatgtaacttactattattagtagtcggtccatgtctattggcccatcatgtggtcattttacgtttacgtcgtgtgactgtttattataacaaacggcacatccttctcattcgaattgtatttctccttaatcgttctaataggtatgatcttttattttatacgtaaaattaaaattgaatgatgtcaagaacgaaaattaatttgtatttacaaaggagctaaatattgtttattcctctactggtagaagataaaagaagtagatgaaataatgatcttactagagaatattcctcatttacactagtcaaatggaaatcttgtaaacttttacaataatttatcccgaaaatatgaaaaaatagaagaaaatgtttacctcctctctcctcttaattcaccttttctattcatttttaatttaaccagtaaaattctataataataataaaaacattttgttacaaaaaattacaatataatgtatataagttctacctaaatttcgtaagtttgttttgagtcgaagaaataaattttctttcaaactttgagatctccgtgtttgagattcttgcagaaatttcagtttgattgaattgcatctatggctcctccatcaaatcatgcgttaactgttaagtataacagtttttaggactcagattaaatgatagatttttttctttctcttaaatagtaaaaattttcttctataagtttcttgttaatttaatttgttttactttcactgaacatacaaaatattcgtttacattttttaatataaaatttttgtttgaactaatcaacatcttaattaataattctacaaattaattccgataaggtctaaatgtcacatgcatagccatttaacatgttaaccaataatctaaactaatttgtatggtacaaacaacagatgtcactgtttctgctcaacctactctttctttttgactaggataattttaaatctatcacaatcatcttcgaataattaaatatctgacgctaacgtgattttgcatttgtaacctttattaatgggccgctaaaagagtttcttatccaaaaaagcaaatattttattaacccaatctctttttcacacaaagataaaatacatcaaagttcacaaacagaggcatcatacaaaaacacaaacattcaaaagcaaaaccccaaatcttcagttcccagaatcttgaactcccttacctccgccactgcctcccaactcacttccaccggaatccgaaaaccccatagtccctaccatagcaaccaagcttatcgataccgtcgacctcgagggggggcccggtaccttaattaaaagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatc

SEQ ID NO: 14 - DNA, 74 bp, Artificial Sequence
  Description of Artificial Sequence Synthetic oligonucleotide
tttgtcgacgtcgacaatggaccacttcttctcttcttactccgatcccagctcctgcagcctcgactttgctg

SEQ ID NO: 15 - DNA, 40 bp, Artificial Sequence
  Description of Artificial Sequence Synthetic oligonucleotide
aaagcggccgcgcggccgcgcatttaaacctaaacaaccc

SEQ ID NO: 16 - DNA, 36 bp, Artificial Sequence
  Description of Artificial Sequence Synthetic oligonucleotide
tttgtcgacgtcgaccatggcagcccccgggaactt

SEQ ID NO: 17 - DNA, 39 bp, Artificial Sequence
  Description of Artificial Sequence Synthetic oligonucleotide
aaagcggccgcgcggccgcgggatggtcaatttatcgtc

SEQ ID NO: 18 - DNA, 9833 bp, Artificial Sequence
  Description of Artificial Sequence Synthetic nucleotide construct
cgggccccccctcgaggtcgacggtatcgataagcttctaatacgactcactataggggtccgatcaaggacatccatccaacgacgcaattccacgtttccccttcctagtggctaggacagcgccacccacctcccctcgcctccctctctctctactactttctctctccttctttttcccaccgtttgaccgtgcctttccagctcccctcgctcgcgtacaccgtaagctcacgcgccatccgacgaaagcggggccgctcgtcgagagcagcgcgggcgcgtgcgttccctcggcgcgtcggcccgtggcgataccctttttgggtcggctgtccgacttcctctcttaacgcaccacgtcggttaagaagcgggagcggattggccgccccctctaggtgggcccacttcagatctctcccagaattccttccttcttcatgcttaatcgcgtcatttccgtgtcaattctatgctataaaaaccgcccaaactcactcctcagcatcatcacacagaaaacccatcgagaaacaccagaaaaagctccgaggaatacgtctgtcgtcttttatcattcgcattaccgcatacttcgaagatcgccggtcgttgatcgcagcagcggtgtgtgttgagagccatggatccagtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagcccgggggatccactagttctagagcggccgcttggcgcgccgtcaacggatcaggatatccttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcacgtaggggggatccactagttctagagcggccgtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagttgtctatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaacttttaattaaggtac

SEQ ID NO: 19 - DNA, 9833 bp, Artificial Sequence
  Description of Artificial Sequence Synthetic nucleotide construct
cgggccccccctcgaggtcgacggtatcgataagcttctaatacgactcactatagggcttagatcaaggacatccatccaacgacgcaattccacgtttccccttcctagtggctaggacagcgccacccacctcccctcgcctccctctctctctactactttctctctccttctttttcccaccgtttgactgtgcctttccagctcccctcgctcgcgtacaccgtaagctcacgcgccatccgacgaaagcggggccgctcgtcgagagcagcgcgggcgcgtgcgttccctcggcgcgtcggcccgtggcgataccctttttgggtcggctgtccgacttcctctcttaacgcaccacgtcggttaagaagcgggagcggattggccgccccctctaggtgggcccacttcagatctctcccagaattccttccttcttcatgcttaatcgcgtcgtttccgcgtcaaatctatgctataaaaaccgcccaaactcactcctcagcatcatcacacagaaaacccatcgagaaacaccagaaaaagctccgaggaatacgtctgtcgtcttttatcattcgcattaccgcatacttcgaagatcgccggtcgttgatcgcagcagcggtgtgtgttgagagccatggatccagtagaaaccccaacccgtgaaatcaaaaaactcgacggcctgtgggcattcagtctggatcgcgaaaactgtggaattggtcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagcccgggggatccactagttctagagcggccgcttggcgcgccgtcaacggatcaggatatccttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcacgtaggggggatccactagttctagagcggccgtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagttgtctatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaacttttaattaaggtac